(19) United States
(12) Patent Application Publication
(10) Pub. No.: US 2008/0319043 A1
(43) Pub. Date: Dec. 25, 2008

(54) 3,6-Disubstituted Azabicyclo (3.1.0) Hexane Derivatives as Muscarinic Receptor Antagonists

(76) Inventors: Mohammad Salman, Princeton, NJ (US); Naresh Kumar, Gurgaon (IN); Kirandeep Kaur, Gurgaon (IN); Shelly Aeron, New Delhi (IN); Pakala Kumara Savithru Sarma, Secunderabad (IN); Sankaranarayanan Dharmarajan, Gurgaon (IN); Anita Mehta, Plainfield, IL (US); Anita Chugh, New Delhi (IN)

Correspondence Address: RANBAXY INC., 600 COLLEGE ROAD EAST, SUITE 2100, PRINCETON, NJ, 08540, US

(21) Appl. No.: 11/913,599

(22) PCT Filed: May 1, 2006
(86) PCT No.: PCT/IB06/51368

§ 371 Date: Jul. 30, 2008

(30) Foreign Application Priority Data
May 3, 2005 (IN) 1810/DELNP/2005
Mar. 28, 2006 (IN) 1681/DELNP/2006

(51) Int. Cl. A61K 31/4035; C07D 209/44

(52) U.S. Cl. 514/412; 548/515

(57) ABSTRACT

The present invention generally relates to muscarinic receptor antagonists, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### FIELD OF THE INVENTION

This invention generally relates to muscarinic receptor antagonists which are useful, among other uses, for the treatment of various diseases of the respiratory, urinary and gastrointestinal systems mediated through muscarinic receptors. Specifically, the invention relates to derivatives of azabicyclo compounds, including, for example, 6-substituted azabicyclo[3.1.0]hexanes, as well as pharmaceutical compositions containing such compounds and methods of treating diseases mediated through muscarinic receptors.

### BACKGROUND OF THE INVENTION

Muscarinic receptors as members of the G Protein Coupled Receptors (GPCRs) are composed of a family of 5 receptor sub-types (M₁, M₂, M₃, M₄ and M₅) and are activated by the neurotransmitter acetylcholine. These receptors are widely distributed on multiple organs and tissues and are critical to the maintenance of central and peripheral cholinergic neurotransmission. The regional distribution of these receptor sub-types in the brain and other organs has been documented. For example, the M₁ subtype is located primarily in neuronal tissues such as cereberal cortex and autonomic ganglia, the M₂ subtype is present mainly in the heart where it mediates cholinergically induced bradycardia, and the M₃ subtype is located predominantly on smooth muscle and salivary glands (Nature, 323, p. 411 (1986); Science, 237, p. 527 (1987)).

A review in Current Opinions in Chemical Biology, 3, p. 426 (1999), as well as in Trends in Pharmacological Sciences, 22, p. 409 (2001) by Eglen et. al., describes the biological potentials of modulating muscarinic receptor subtypes by ligands in different disease conditions, such as Alzheimer's Disease, pain, urinary disease condition, chronic obstructive pulmonary disease, and the like.

A review in J. Med. Chem., 43, p. 4333 (2000), by Felder et. al. describes therapeutic opportunities for muscarinic receptors in the central nervous system and elaborates on muscarinic receptor structure and function, pharmacology and their therapeutic uses.

The pharmacological and medical aspects of the muscarinic class of acetylcholine agonists and antagonists are presented in a review in Molecules, 6, p. 142 (2001). Birdsall et. al. in Trends in Pharmacological Sciences, 22, p. 215 (2001) have also summarized the recent developments on the role of different muscarinic receptor subtypes using different muscarinic receptor of knock out mice.

Muscarinic agonists such as muscarine and pilocarpine and antagonists such as atropine have been known for over a century, but little progress has been made in the discovery of receptor subtype-selective compounds, making it difficult to assign specific functions to the individual receptors. Although classical muscarinic antagonists such as atropine are potent bronchodilators, their clinical utility is limited due to high incidence of both peripheral and central adverse effects such as tachycardia, blurred vision, dryness of mouth, constipation, dementia, etc. Subsequent development of the quarterly derivatives of atropine such as ipratropium bromide are better tolerated than parenterally administered options, but most of these are not ideal anti-cholinergic bronchodilators, due to lack of selectivity for muscarinic receptor sub-types, resulting in dose-limiting side-effects such as thirst, nausea, mydriasis and those associated with the heart such as tachycardia mediated by the M₂ receptor.

Annual Review of Pharmacological Toxicol., 41, p. 691 (2001), describes the pharmacology of the lower urinary tract infections. Although anti-muscarinic agents such as oxybutynin and tolterodine that act non-selectively on muscarinic receptors have been used for many years to treat bladder hyperactivity, the clinical effectiveness of these agents has been limited due to the side effects such as dry mouth, blurred vision and constipation. Tolterodine is considered to be generally better tolerated than oxybutynin. (Steers et. al., in Curr. Opin. Invest. Drugs, 2, 268; Chapple et. al., in Urology, 55, 33; Steers et al., Adult and Pediatric Urology, ed. Gillenwatter et al., pp 1220-1325, St. Louis, Mo.; Mosby. 3d edition (1996)).

There remains a need for development of new highly selective muscarinic antagonists, which can interact with distinct subtypes, thus avoiding the occurrence of adverse effects.

Compounds reported as having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 97/45414 are related to 1,4-disubstituted piperidine derivatives; WO 98/05641 describes fluorinated, 1,4-disubstituted piperidine derivatives; WO 93/16018 and WO96/33973 are other references of interest.

A report in J. Med. Chem., 44, p. 984 (2002), describes cyclohexylmethyl piperidinyl triphenylpropioamide derivatives as selective M₃ antagonist discriminating against the other receptor subtypes.

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I:

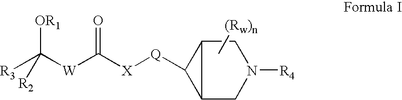

and their pharmaceutically acceptable salts, pharmaceutically acceptable solvates, esters, enantiomers, diastereomers, N-oxides, polymorphs, metabolites, wherein
R₁ is hydrogen or alkyl;
R₂ is straight or branched alkyl alkenyl, alkynyl, aryl, cycloalkyl, cycloalkylalkyl or heteroaryl wherein the alkyl, alkenyl, alkynyl, aryl, cycloalkyl, cycloalkylalkyl and heteroaryl are optionally substituted with one or more substituents selected from alkyl, hydroxy or halogen.
R₃ is aryl or heteroaryl wherein the said aryl or heteroaryl are optionally substituted with one or more groups selected from alkyl, hydroxy or halogen;
W=—(CH₂)ᵢ;
Q=—(CH₂)ⱼ;
X is oxygen or —N(R₅)—;
R₄ is hydrogen, straight or branched alkyl, straight or branched alkenyl, aralkyl or heteroarylalkyl wherein the said aralkyl or heteroarylalkyl is further substituted with alkyl, —NH₂ or alkoxycarbonylamino;
R₅ is hydrogen or alkyl;
R_{w} is H or methyl; and
n, i, j are integer from 0-2.

In accordance with a second aspect, there is provided a method for treatment or prophylaxis of a disease or disorder of the respiratory, urinary and gastrointestinal systems in an animal or a human suffering therefrom, wherein the disease or disorder is mediated through muscarinic receptors. The method includes administration of at least one compound having the structure of Formula I.

In accordance with a third aspect, there is provided a method for treatment or prophylaxis of an animal or a human suffering from a disease or disorder associated with muscarinic receptors, comprising administering to a patient in need thereof, an effective amount of a muscarinic receptor antagonist compound as described above.

In accordance with a fourth aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fifth aspect, there are provided processes for preparing the compounds as described above.

The compounds described herein exhibit significant potency in terms of their activity, as determined by in vitro receptor binding and functional assays and in vivo experiments using anaesthetized rabbits. The compounds that were found active in vitro were tested in vivo. Some of the compounds were found to be potent muscarinic receptor antagonists with high affinity towards M₃ receptors. Therefore, pharmaceutical compositions for the possible treatment for the disease or disorders associated with muscarinic receptors are provided. In addition, the compounds can be administered orally or parenterally.

### DETAILED DESCRIPTION OF THE INVENTION

The compounds of the present invention may be prepared by techniques well known in the art and familiar to a practitioner skilled in art of this invention. In addition, although the compounds of the present invention may be prepared by processes described herein, such processes are not the only means by which the compounds described may be synthesised. Further, the various synthetic steps described herein may be performed in an alternate sequence in order to give the desired compounds.

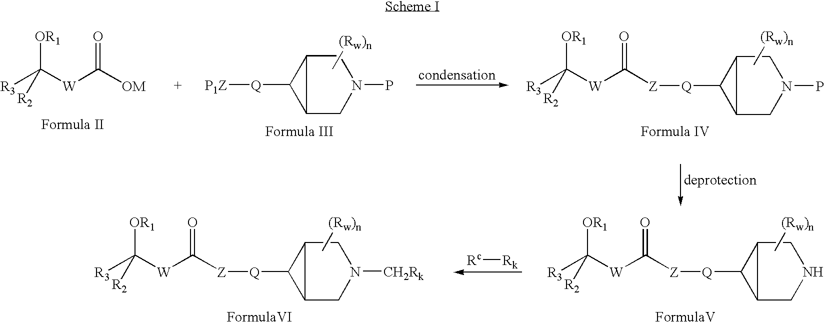

The compounds of Formula IV, V and VI can be prepared by the reaction procedure as depicted, for example, in Scheme I, thus a compound of Formula II (wherein M is alkyl or hydrogen; R₁, R₂, R₃ and W are the same as defined earlier) can be reacted with a compound of Formula III (wherein Z is oxygen or —NR₅ (wherein R₅ is the same as defined earlier); P₁ is hal (CT, Br or I), mesyl, tosyl or H; Q, R_{w} and n are the same as defined earlier and P is benzyl, —C(═O)OtBu or —C(═O)OCH₂C₆H₅) to give a compound of Formula IV, which can undergo deprotection to give a compound of Formula V, which can undergo N-derivatization with a compound of Formula R^{c}—Rₖ [wherein R^{c} is CHO or CH₂hal (hal is Br, Cl or I) and Rₖ is hydrogen, alkyl, alkenyl, aryl, aralkyl, heteroarylalkyl, or heteroaryl] to give a compound of Formula VI.

The reaction of a compound of Formula II (when M is hydrogen) with a compound of Formula III (when Z is —NR₅ and P, is H) to give a compound of Formula IV can be carried out in an organic solvent, for example, dimethylformamide, tetrahydrofuran, dioxane, chloroform or diethylether, in the presence of a base, for example, N-methylmorpholine, pyridine, triethylamine or diisopropylethylamine, with a condensing agent, for example, 1-(3-dimethylaminopropyl)-3-ethyl carbodiimide hydrochloride or dicyclohexylcarbodiimide.

The reaction of a compound of Formula II (when M is hydrogen) with a compound of Formula III (when Z is oxygen and P₁ is hal (Br, Cl or I), mesyl or tosyl) to give a compound of Formula IV can be carried out in an organic solvent, for example, toluene, benzene or xylene, in the presence of a base, for example, 1,8-diazabicyclo[5.4.0]undec-7-ene, N-methylmorpholine, triethylamine or diisopropylethylamine.

The reaction of a compound of Formula II (when M is alkyl) with a compound of Formula III (when Z is oxygen and P₁ is H) to give a compound of Formula IV can be carried out in an organic solvent, for example, tetrahydrofuran, diethyl ether, dioxane or dimethylformamide in the presence of a base, for example, butyl lithium, lithium diisopropyl amide, sodium hydride or diisopropylethylamine.

The reaction of a compound of Formula II (when M is alkyl) with a compound of Formula III (when Z is —NR₅ and P₁ is H) to give a compound of Formula IV can be carried out in an organic solvent, for example, tetrahydrofuran, diethyl ether, dioxane or dimethylformamide, in the presence of a reducing agent, for example, diisobutyl aluminum.

The deprotection of a compound of Formula IV (when P is benzyl) to give a compound of Formula V can be carried out in an organic solvent, for example, methanol, ethanol, propanol or isopropylalcohol, with a deprotecting agent, for example, palladium on carbon in the presence of hydrogen gas or palladium on carbon with a source of hydrogen gas such as ammonium formate, cyclohexene or formic acid.

The deprotection of a compound of Formula IV (when P is —C(═O)OCH₂C₆H₅) to give a compound of Formula V can be carried out in an organic solvent, for example, methanol, ethanol, propanol or isopropylalcohol, in the presence of methanolic or ethanolic potassium hydroxide.

The deprotection of a compound of Formula IV (when P is —C(═O)OtBu) to give a compound of Formula V can be carried out with a strong acid, for example, hydrochloric acid, trifluoroacetic acid in an organic solvent, for example, methanol, ethanol, propanol, isopropylalcohol, diethylether, tetrahydrofuran or dichloromethane.

The compound of Formula V is reacted with Rₖ—R^{c} (when R_{c} is —CHO) to give a compound of Formula VI in an organic solvent for example, acetonitrile, dichloromethane, dichloroethane, tetrahydrofuran, dioxane, chloroform or carbon tetrachloride, in the presence of, for example, sodium cyanoborohydride or sodium triacetoxyborohydride.

The compound of Formula V is reacted with Rₖ—R^{c} (when R_{c} is —CH₂hal) to give a compound of Formula VI in an organic solvent, for example, dimethylformamide acetonitrile, dichloromethane, dichloroethane or chloroform, in the presence of a base, for example, potassium carbonate, sodium carbonate, lithium carbonate, potassium bicarbonate or sodium bicarbonate.

Illustrative compounds which can be prepared following, for example, Scheme I include:
N-(3-Benzyl-3-azabicyclo[3.1.0]hex-6-yl)-2-hydroxy-2-phenyl-2-(2-thienyl)acetamide (Compound No. 1),
N-[(3-Benzyl-3-azabicyclo[3.1.0]hex-6-yl)methyl]-2-hydroxy-2-phenyl-2-(2-thienyl)acetamide (Compound No. 2),
(3-Benzyl-3-azabicyclo[3.1.0]hex-6-yl)methyl hydroxy(phenyl)-2-thienylacetate (Compound No. 3),
N-(3-Benzyl-3-azabicyclo[3.1.0]hex-6-yl)-2-hydroxy-2-phenyl-2-(3-thienyl)acetamide (Compound No. 5),
N-[(3-Benzyl-3-azabicyclo[3.1.0]hex-6-yl)methyl]-2-hydroxy-2-phenyl-2-(3-thienyl)acetamide (Compound No. 6),
Tartarate salt of N-[(3-benzyl-3-azabicyclo[3.1.0]hex-6-yl)methyl]-2-cyclopentyl-2-hydroxy-2-(2-thienyl)acetamide (Compound No. 7),
N-[(3-Benzyl-3-azabicyclo[3.1.0]hex-6-yl)methyl]-2-hydroxy-N-methyl-2-phenyl-2-(3-thienyl)acetamide (Compound No. 8),
Tartarate salt of N-(3-benzyl-3-azabicyclo[3.1.0]hex-6-yl)-2-cyclopentyl-2-hydroxy-2-(2-thienyl)acetamide (Compound No. 9),
N-[(3-Benzyl-3-azabicyclo[3.1.0]hex-6-yl)methyl]-2-hydroxy-2,2-di-2-thienylacetamide (Compound No. 10),
N-[(3-Benzyl-3-azabicyclo[3.1.0]hex-6-yl)methyl]-2-hydroxy-N-methyl-2-phenyl-2-(2-thienyl)acetamide (Compound No. 11),
N-[(3-Benzyl-3-azabicyclo[3.1.0]hex-6-yl)methyl]-2-cyclopentyl-2-hydroxy-N-methyl-2-(2-thienyl)acetamide (Compound No. 12),
N-[(3-Benzyl-3-azabicyclo[3.1.0]hex-6-yl)methyl]-2-hydroxy-N-methyl-2-(4-methylphenyl)-2-(2-thienyl)acetamide (Compound No. 13),
(3-Benzyl-3-azabicyclo[3.1.0]hex-6-yl)methyl hydroxy(4-methylphenyl)-2-thienylacetate (Compound No. 14),
(3-Benzyl-3-azabicyclo[3.1.0]hex-6-yl)methyl hydroxy(di-2-thienyl)acetate (Compound No. 15),
N-[(3-Benzyl-3-azabicyclo[3.1.0]hex-6-yl)methyl]-2-hydroxy-2-(4-methylphenyl)-2-(2-thienyl)acetamide (Compound No. 16),
Tartarate salt of N-(3-azabicyclo[3.1.0]hex-6-ylmethyl)-2-cyclopentyl-2-hydroxy-2-(2-thienyl)acetamide (Compound No. 17),
N-(3-Azabicyclo[3.1.0]hex-6-ylmethyl)-2-hydroxy-2-phenyl-2-(2-thienyl)acetamide (Compound No. 18),
2-Hydroxy-N-[(3-methyl-3-azabicyclo[3.1.0]hex-6-yl)methyl]-2-phenyl-2-(2-thienyl)acetamide (Compound No. 19),
N-(3-Benzyl-3-azabicyclo[3.1.0]hex-6-yl)-2-(4-fluorophenyl)-2-hydroxy-2-phenylacetamide (Compound No. 20),
N-(3-Benzyl-3-azabicyclo[3.1.0]hex-6-yl)-2-hydroxy-2-(4-methylphenyl)-2-phenylacetamide (Compound No. 21),
(3-Benzyl-3-azabicyclo[3.1.0]hex-6-yl)methyl 3-hydroxy-3,3-diphenylpropanoate (Compound No. 22),
N-[(3-Benzyl-3-azabicyclo[3.1.0]hex-6-yl)methyl]-3-hydroxy-N-methyl-3,3-diphenylpropanamide (Compound No. 23),
N-[(3-Benzyl-3-azabicyclo[3.1.0]hex-6-yl)methyl]-3-hydroxy-3,3-diphenylpropanamide (Compound No. 24),
(3-Methyl-3-azabicyclo[3.1.0]hex-6-yl)methyl hydroxy(3-methylphenyl)phenylacetate (Compound No. 25),
N-[(3-Benzyl-3-azabicyclo[3.1.0]hex-6-yl)methyl]-2-hydroxy-2-(2-hydroxy-5-methylphenyl)-2-phenylacetamide (Compound No. 26),
3-Azabicyclo[3.1.0]hex-6-ylmethyl hydroxy[bis(3-methylphenyl)]acetate (Compound No. 27),
N-(3-Azabicyclo[3.1.0]hex-6-ylmethyl)-2-hydroxy-2,2-bis(3-methylphenyl)acetamide (Compound No. 28),
N-[(3-Benzyl-3-azabicyclo[3.1.0]hex-6-yl)methyl]-2-hydroxy-2,2-bis(3-methylphenyl)acetamide (Compound No. 29),
N-(3-Azabicyclo[3.1.0]hex-6-ylmethyl)-2-hydroxy-N-methyl-2,2-bis(3-methylphenyl)acetamide (Compound No. 30),
N-[(3-Benzyl-3-azabicyclo[3.1.0]hex-6-yl)methyl]-2-methoxy-2,2-diphenylacetamide (Compound No. 32),
N-(3-Benzyl-3-azabicyclo[3.1.0]hex-6-yl)-2-methoxy-2,2-diphenylacetamide (Compound No. 33),
N-[(3-Benzyl-3-azabicyclo[3.1.0]hex-6-yl)methyl]-2-methoxy-N-methyl-2,2-diphenylacetamide (Compound No. 34),
N-(3-Azabicyclo[3.1.0]hex-6-ylmethyl)-3-hydroxy-3,3-diphenylpropanamide (Compound No. 36),
Tartarate salt of N-(3-azabicyclo[3.1.0]hex-6-ylmethyl)-3-hydroxy-N-methyl-3,3-diphenylpropanamide (Compound No. 37),
N-(3-Azabicyclo[3.1.0]hex-6-ylmethyl)-2-methoxy-N-methyl-2,2-diphenylacetamide (Compound No. 38),
N-3-Azabicyclo[3.1.0]hex-6-yl-2-methoxy-2,2-diphenylacetamide (Compound No. 39),
N-(3-Azabicyclo[3.1.0]hex-6-ylmethyl)-2-methoxy-2,2-diphenylacetamide (Compound No. 40),
N-(3-Azabicyclo[3.1.0]hex-6-ylmethyl)-2-methoxy-2-(4-methylphenyl)-2-phenylacetamide (Compound No. 41),
3-Azabicyclo[3.1.0]hex-6-ylmethyl diphenyl(propoxy)acetate (Compound No. 42),
N-(3-Azabicyclo[3.1.0]hex-6-ylmethyl)-N-methyl-2,2-diphenyl-2-propoxyacetamide (Compound No. 43),
N-(3-Azabicyclo[3.1.0]hex-6-ylmethyl)-2,2-diphenyl-2-propoxyacetamide (Compound No. 44),
2-Hydroxy-N-methyl-2,2-diphenyl-N-{[3-(2-thienylmethyl)-3-azabicyclo[3.1.0]hex-6-yl]methyl}acetamide (Compound No. 45),
3-Azabicyclo[3.1.0]hex-6-ylmethyl hydroxy[bis(4-methylphenyl)]acetate (Compound No. 46),
N-(3-Azabicyclo[3.1.0]hex-6-ylmethyl)-2,2-bis(4-fluorophenyl)-2-hydroxy-N-methylacetamide (Compound No. 47),
N-(3-Azabicyclo[3.1.0]hex-6-ylmethyl)-2-hydroxy-N-methyl-2,2-bis(4-methylphenyl)acetamide (Compound No. 48),
N-(3-benzyl-3-azabicyclo[3.1.0]hex-6-yl)-N-ethyl-2-hydroxy-2,2-diphenylacetamide (Compound No. 49)
2-Cyclohexyl-2-hydroxy-N-({3-[(6-methylpyridin-2-yl)methyl-3-azabicyclo[3.1.0]hex-6-yl}methyl)-2-phenylacetamide (Compound No. 50),
2-Cycloheptyl-2-hydroxy-2-phenyl-N-{[3-(pyridin-2-ylmethyl)-3-azabicyclo[3.1.0]hex-6-yl]methyl}acetamide (Compound No. 55),
2-Cyclopentyl-2-hydroxy-N-({3-[(6-methylpyridin-2-yl)methyl]-3-azabicyclo[3.1.0]hex-6-yl}methyl)-2-phenylacetamide (Compound No. 56),
(3-Benzyl-2-methyl-3-azabicyclo[3.1.0]hex-6-yl)methyl cyclopentyl(hydroxy)phenylacetate (Compound No. 58),
(3-Benzyl-2-methyl-3-azabicyclo[3.1.0]hex-6-yl)methyl cyclohexyl(hydroxy)phenylacetate (Compound No. 59),
N-[(3-Benzyl-2-methyl-3-azabicyclo[3.1.0]hex-6-yl)methyl]-2-cyclopentyl-2-hydroxy-2-phenylacetamide (Compound No. 60),
N-[(3-Benzyl-2-methyl-3-azabicyclo[3.1.0]hex-6-yl)methyl]-2-cyclohexyl-2-hydroxy-2-phenylacetamide (Compound No. 61),
(3-Benzyl-2-methyl-3-azabicyclo[3.1.0]hex-6-yl)methyl hydroxy(diphenyl)acetate (Compound No. 62),
Tartarate salt of N-[(3-benzyl-2-methyl-3-azabicyclo[3.1.0]hex-6-yl)methyl]-2-cyclohexyl-2-hydroxy-2-phenylacetamide (compound No. 63),
(2-Methyl-3-azabicyclo[3.1.0]hex-6-yl)methyl cyclohexyl(hydroxy)phenylacetate (Compound No. 64),
Tartarate salt of [3-(4-methylpent-3-en-1-yl)-3-azabicyclo[3.1.0]hex-6-yl]methyl cyclopentyl(hydroxy)phenylacetate (Compound No. 68),
Tartarate salt of [3-(1-phenylethyl)-3-azabicyclo[3.1.0]hex-6-yl]methyl cyclohexyl(hydroxy)phenylacetate (Compound No. 69),
Tartarate salt of N-[(3-benzyl-3-azabicyclo[3.1.0]hex-6-yl)methyl]-2-cyclobutyl-2-hydroxy-2-phenylacetamide (Compound No. 70),
2-Hydroxy-N-{[3-(3-methylbut-2-en-1-yl)-3-azabicyclo[3.1.0]hex-6-yl]methyl}-2,2-diphenylacetamide (Compound No. 71),
2-Hydroxy-N-{[3-(4-methylpent-3-en-1-yl)-3-azabicyclo[3.1.0]hex-6-yl]methyl}-2,2-diphenylacetamide (Compound No. 72),
2-Cycloheptyl-2-hydroxy-2-phenyl-N-{[3-(pyridin-3-ylmethyl)-3-azabicyclo[3.1.0]hex-6-yl]methyl}acetamide (Compound No. 73),
2-Cycloheptyl-2-hydroxy-2-phenyl-N-{[3-(pyridin-4-ylmethyl)-3-azabicyclo[3.1.0]hex-6-yl]methyl}acetamide (Compound No. 74),
2-Cyclopentyl-2-hydroxy-N-[(3-methyl-3-azabicyclo[3.1.0]hex-6-yl)methyl]-2-phenylacetamide (Compound No. 75),
2-Cyclopentyl-2-hydroxy-2-phenyl-N-{[3-(2-phenylethyl)-3-azabicyclo[3.1.0]hex-6-yl]methyl}acetamide (Compound No. 76),
N-[(3-Benzyl-3-azabicyclo[3.1.0]hex-6-yl)methyl]-2-cycloheptyl-2-hydroxy-2-phenylacetamide (Compound No. 77),
N-[(3-Benzyl-3-azabicyclo[3.1.0]hex-6-yl)methyl]-2-cyclohexyl-2-hydroxy-N-methyl-2-phenylacetamide (Compound No. 78),
N-[(3-Benzyl-3-azabicyclo[3.1.0]hex-6-yl)methyl]-2-hydroxy-N-methyl-2,2-diphenylacetamide (Compound No. 79),
N-{[3-(1,3-Benzodioxol-5-ylmethyl)-3-azabicyclo[3.1.0]hex-6-yl]methyl}-2-cyclohexyl-2-hydroxy-2-phenylacetamide (Compound No. 80),
2-Cyclopentyl-2-hydroxy-2-phenyl-N-{[3-(2-thienylmethyl)-3-azabicyclo[3.1.0]hex-6-yl]methyl}acetamide (Compound No. 81),
2-Cyclohexyl-2-hydroxy-2-phenyl-N-{[3-(2-phenylethyl)-3-azabicyclo[3.1.0]hex-6-yl]methyl}acetamide (Compound No. 82),
N-({3-[2-(1,3-Benzodioxol-5-yl)ethyl]-3-azabicyclo[3.1.0]hex-6-yl}methyl)-2-cycloheptyl-2-hydroxy-2-phenylacetamide (Compound No. 83),
2-Cycloheptyl-N-({3-[2-(2,3-dihydro-1-benzofuran-5-yl)ethyl]-3-azabicyclo[3.1.0]hex-6-yl}methyl)-2-hydroxy-2-phenylacetamide (Compound No. 84),
N-[(3-Benzyl-3-azabicyclo[3.1.0]hex-6-yl)methyl]-2-hydroxy-3-methyl-2-phenylpentanamide (Compound No. 85),
N-[(3-Benzyl-3-azabicyclo[3.1.0]hex-6-yl)methyl]-2-cyclopentyl-2-methoxy-2-phenylacetamide (Compound No. 86),
Tartarate salt of [3-(3-methylbut-2-en-1-yl)-3-azabicyclo[3.1.0]hex-6-yl]methyl cyclopentyl(hydroxy)phenylacetate (Compound No. 87),
[3-(1,3-Benzodioxol-5-ylmethyl)-3-azabicyclo[3.1.0]hex-6-yl]methyl cyclopentyl (hydroxy)phenylacetate (Compound No. 88),
Tartarate salt of N-[(3-benzyl-3-azabicyclo[3.1.0]hex-6-yl)methyl]-2-cyclohexyl-2-hydroxy-N-methyl-2-phenylacetamide (Compound No. 89),
Tartarate salt of N-[(3-benzyl-3-azabicyclo[3.1.0]hex-6-yl)methyl]-2-hydroxy-N-methyl-2,2-diphenylacetamide (Compound No. 90),
N-[(3-Benzyl-3-azabicyclo[3.1.0]hex-6-yl)methyl]-3-cyclohexyl-2-hydroxy-2-phenylpropanamide (Compound No. 91),
2-Cyclopentyl-N-{[3-(cyclopropylmethyl)-3-azabicyclo[3.1.0]hex-6-yl]methyl}-2-hydroxy-2-phenylacetamide (Compound No. 92),
N-(3-Benzyl-3-azabicyclo[3.1.0]hex-6-yl)-2-cyclohexyl-N-ethyl-2-hydroxy-2-phenylacetamide (Compound No. 93),
N-3-azabicyclo[3.1.0]hex-6-yl-2-cyclohexyl-N-ethyl-2-hydroxy-2-phenylacetamide (Compound No. 94),
Tartarate salt of (3-benzyl-3-azabicyclo[3.1.0]hex-6-yl)methyl cyclopentyl(hydroxy)-2-thienylacetate (Compound No. 95),
N-[(3-Benzyl-3-azabicyclo[3.1.0]hex-6-yl)methyl]-3,3,3-trifluoro-2-hydroxy-2-phenylpropanamide (Compound No. 96),
(3-Benzyl-3-azabicyclo[3.1.0]hex-6-yl)methyl 2-hydroxy-2-phenylpent-4-ynoate (Compound No. 97),
N-[(3-Benzyl-3-azabicyclo[3.1.0]hex-6-yl)methyl]-2-hydroxy-2-phenylpent-4-ynamide (Compound No. 98),
[3-(1,3-Benzodioxol-5-ylmethyl)-3-azabicyclo[3.1.0]hex-6-yl]methyl cyclohexyl(hydroxy)phenylacetate (Compound No. 99),
2-Hydroxy-2,2-diphenyl-N-{[3-(2-phenylethyl)-3-azabicyclo[3.1.0]hex-6-yl]methyl}acetamide (Compound No. 100),
N-[(3-Benzyl-3-azabicyclo[3.1.0]hex-6-yl)methyl]-2-hydroxy-3-methyl-2-phenylbutanamide (Compound No. 101),
N-({3-[2-(1,3-Benzodioxol-5-yl)ethyl]-3-azabicyclo[3.1.0]hex-6-yl}methyl)-2-cyclopentyl-2-hydroxy-N-methyl-2-phenylacetamide (Compound No. 102),
Tartarate salt of N-[(3-Benzyl-3-azabicyclo[3.1.0]hex-6-yl)methyl]-2-cyclopentyl-2-hydroxy-N-methyl-2-phenylacetamide (Compound No. 103),
N-[(3-Benzyl-3-azabicyclo[3.1.0]hex-6-yl)methyl]-2-hydroxy-2-phenyl-2-pyridin-2-ylacetamide (Compound No. 104),
N-[(3-Benzyl-3-azabicyclo[3.1.0]hex-6-yl)methyl]-2-hydroxy-N-methyl-2-phenyl-2-pyridin-3-ylacetamide (Compound No. 105),
2-Hydroxy-N-methyl-N-{[3-(4-methylpent-3-en-1-yl)-3-azabicyclo[3.1.0]hex-6-yl]methyl}-2,2-diphenylacetamide (Compound No. 106),
N-({3-[2-(1,3-Benzodioxol-5-yl)ethyl]-3-azabicyclo[3.1.0]hex-6-yl}methyl)-2-hydroxy-N-methyl-2,2-diphenylacetamide (Compound No. 107),
N-({3-[2-(2,3-Dihydro-1-benzofuran-5-yl)ethyl]-3-azabicyclo[3.1.0]hex-6-yl}methyl)-2-hydroxy-N-methyl-2,2-diphenylacetamide (Compound No. 108),
N-{3-Azabicyclo[3.1.0]hex-6-ylmethyl}-N-methyl-2,2-diphenylpropanamide (Compound No. 109),
N-[(3-Benzyl-3-azabicyclo[3.1.0]hex-6-yl)methyl]-2-hydroxy-N-methyl-2-phenylhex-4-enamide (Compound No. 110),
N-[(3-Benzyl-3-azabicyclo[3.1.0]hex-6-yl)methyl]-2-hydroxy-2-phenyl-2-pyridin-3-ylacetamide (Compound No. 111),
N-[(3-Benzyl-3-azabicyclo[3.1.0]hex-6-yl)methyl]-2-hydroxy-2-phenylhex-4-enamide (Compound No. 112),
N-[(3-Benzyl-3-azabicyclo[3.1.0]hex-6-yl)methyl]-N-methyl-2,2-diphenyl-2-propoxyacetamide (Compound No. 113),
3-Azabicyclo[3.1.0]hex-6-ylmethyl (4E&4Z)-2-hydroxy-2-phenylhex-4-enoate (Compound No. 114),
(3-Benzyl-3-azabicyclo[3.1.0]hex-6-yl)methyl (4E&4Z)-2-hydroxy-2-phenylhex-4-enoate (Compound No. 115),
(3-Benzyl-3-azabicyclo[3.1.0]hex-6-yl)methyl (2R or 2S)-hydroxy(4-methylphenyl)phenylacetate (Compound No. 116),
(3-Benzyl-3-azabicyclo[3.1.0]hex-6-yl)methyl hydroxy(phenyl)pyridin-2-ylacetate (Compound No. 117),
(3-Benzyl-3-azabicyclo[3.1.0]hex-6-yl)methyl hydroxy(phenyl)pyridin-3-ylacetate (Compound No. 118),
N-[(3-Benzyl-3-azabicyclo[3.1.0]hex-6-yl)methyl]-N-methyl-2,2-diphenylpropanamide (Compound No. 119), and
Tert-butyl 6-({[(4E)-2-hydroxy-2-phenylhex-4-enoyl]oxy}methyl)-3-azabicyclo[3.1.0]hexane-3-carboxylate (Compound No. 120).

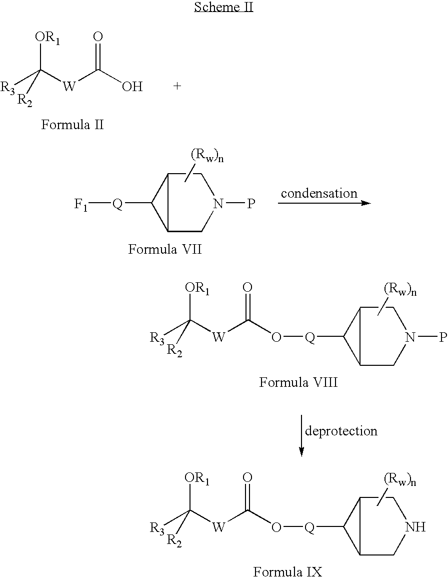

The compound of Formula IX can be prepared by following the procedure as depicted in, for example, Scheme II. Thus a compound of Formula II can be reacted with a compound of Formula VII (wherein F₁ is mesyl, tosyl, triflyl or hal (Br, Cl or I); Q, R_{w}, n and P are the same as defined earlier) to give a compound of Formula VIII, which can undergo deprotection to give a compound of Formula IX.

The reaction of a compound of Formula II with a compound of Formula VII to give a compound of Formula VIII can be carried out in an organic solvent, for example, toluene or xylene in the presence of a base, for example, N-methylmorpholine, pyridine, triethylamine or diisopropylethylamine.

The deprotection of a compound of Formula VIII to give a compound of Formula IX can be carried out following the procedure as described, for example, for the synthesis of compound of Formula V from a compound of Formula IV in Scheme I.

Illustrative compounds which can be prepared following, for example, Scheme II include:
(3-Benzyl-3-azabicyclo[3.1.0]hex-6-yl)methyl hydroxy(phenyl)-3-thienylacetate (Compound No. 4),
(3-Benzyl-3-azabicyclo[3.1.0]hex-6-yl)methyl methoxy(diphenyl)acetate (Compound No. 31), and
3-Azabicyclo[3.1.0]hex-6-ylmethyl methoxy(diphenyl)acetate (Compound No. 35).

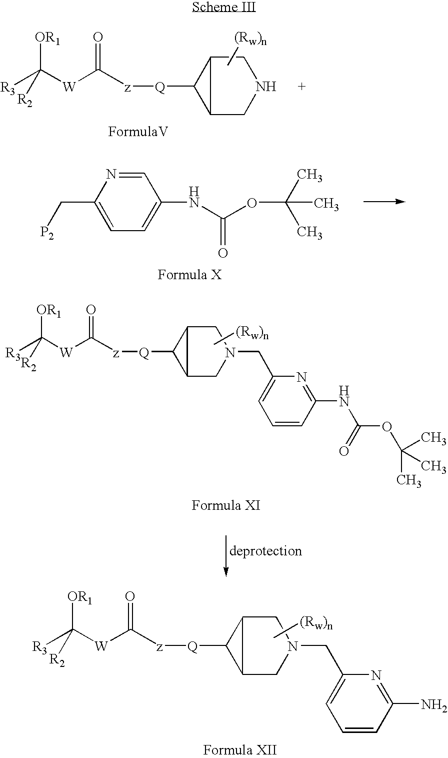

The compounds of Formula XI can be prepared by following, for example, the reaction procedure as depicted in Scheme III. Thus a compound of Formula V can be reacted with a compound of Formula X (wherein P₂ is —Omesyl or —Otosyl) to give a compound of Formula XI (wherein R₁, R₂, R₃, Z, Q, R_{w} and n are the same as defined earlier), which can be deprotected to give a compound of Formula XII.

The reaction of a compound of Formula V with a compound of Formula X to give a compound of Formula XI can be carried out in an organic solvent, for example, acetonitrile, dichloromethane, carbon tetrachloride or chloroform, in the presence of a base, for example, potassium carbonate, sodium carbonate, potassium bicarbonate or sodium bicarbonate.

The deprotection of a compound of Formula XI to give a compound of Formula XII can be carried out with a strong acid, for example, hydrochloric acid, trifluoroacetic acid, in an organic solvent, for example, methanol, ethanol, propanol, isopropylalcohol, diethylether, tetrahydrofuran or dichloromethane.

Illustrative compounds which can be prepared following, for example, Scheme III include:
N-({3-[(6-Aminopyridin-2-yl)methyl]-3-azabicyclo[3.1.0]hex-6-yl}methyl)-2-cyclopentyl-2-hydroxy-2-phenylacetamide (Compound No. 51),
N-({3-[(6-Aminopyridin-2-yl)methyl]-3-azabicyclo[3.1.0]hex-6-yl}methyl)-2-cyclohexyl-2-hydroxy-2-phenylacetamide (compound No. 52),
Tartarate salt of N-({3-[(6-aminopyridin-2-yl)methyl]-3-azabicyclo[3.1.0]hex-6-yl}methyl)-2-cyclopentyl-2-hydroxy-2-phenylacetamide (compound No. 53),
Tartarate salt of N-({3-[(6-aminopyridin-2-yl)methyl]-3-azabicyclo[3.1.0]hex-6-yl}methyl)-2-cyclohexyl-2-hydroxy-2-phenylacetamide (Compound No. 54),
N-({3-[(6-Aminopyridin-2-yl)methyl]-3-azabicyclo[3.1.0]hex-6-yl}methyl)-2-hydroxy-2,2-diphenylacetamide (Compound No. 57),
Tert-butyl (6-{[6-({[cyclohexyl(hydroxy)phenylacetyl]amino}methyl)-3-azabicyclo[3.1.0]hex-3-yl}methyl]pyridin-2-yl)carbamate (Compound No. 65),
Tert-butyl (6-{[6-({[hydroxy(diphenyl)acetyl]amino}methyl)-3-azabicyclo[3.1.0]hex-3-yl]methyl}pyridin-2-yl)carbamate (compound no. 66), and
Tert-butyl (6-{[6-({[cyclopentyl(hydroxy)phenylacetyl]amino}methyl)-3-azabicyclo[3.1.0]hex-3-yl]methyl}pyridin-2-yl)carbamate (Compound No. 67).

In the above scheme, where specific bases, condensing agents, protecting groups, deprotecting agents, solvents, catalysts, temperatures, etc. are mentioned, it is to be understood that other bases, condensing agents, protecting groups, deprotecting agents, solvents, catalysts, temperatures, etc. known to those skilled in the art may be used. Similarly, the reaction temperature and duration may be adjusted according to the desired needs.

Suitable salts of the compounds represented by the Formula I were prepared so as to solubilize the compound in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). When carboxyl groups are included in the Formula I as substituents, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent.

Because of their valuable pharmacological properties, the compounds described herein may be administered to an animal for treatment orally, or by a parenteral route. The pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds described herein can be produced and formulated as their enantiomers, diastereomers, N-Oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

The examples mentioned below demonstrate general synthetic procedures, as well as specific preparations of particular compounds. The examples are provided to illustrate the details of the invention and should not be constrained to limit the scope of the present invention.

### Experimental

Various solvents, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz instrument using tetramethylsilane as an internal standard.

### General Procedure

### Synthesis of Ethyl phenyl (hydroxy)-2-pyridylacetate

A solution of 2-bromo pyridine (3 g, 18.98 mmol) in diethylether (50 ml) was cooled at −78° C. followed by the addition of n-butyl lithium. The reaction mixture was stirred at room temperature for 50 minutes. The resulting reaction mixture was added to a solution of ethyl oxo(phenyl)acetate (3.71 g, 20.88 mmol) in diethyl ether (50 ml) which was cooled at −78° C. The reaction mixture was stirred at same temperature for 40 minutes which was subsequently warmed at room temperature. The mixture was stirred at room temperature for overnight, which was subsequently quenched with ammonium chloride and extracted with ethyl acetate. The organic layer was dried over anhydrous sodium sulphate, filtered and concentrated under reduced pressure. The residue thus obtained was purified by column chromatography using 12% ethyl acetate in hexane solvent mixture as eluent to furnish the title compound. Yield: 1.2 g.

### Synthesis of cyclopentyl (hydroxy)-2-thienylacetic acid

### Step a: Ethyl oxo(2-thienyl)acetate

A solution of thiophene (3 g, 35.65 mmol), ethyl oxalylchloride (4.86 g, 35.65 mmol) in dichloromethane (100 ml) was cooled in an ice bath to −5° C. A suspension of aluminum chloride (4.75 g, 35.65 mmol) in dichloromethane (100 ml) was added dropwise over a period of ½ hour to the reaction mixture and stirred for 1 hour at room temperature. To it was added dilute hydrochloric acid (100 ml). The organic layer was separated, washed with water, dried over anhydrous sodium sulphate and concentrated under reduced pressure. The residue thus obtained was purified by column chromatography using 2% ethyl acetate in hexane solvent mixture to furnish the title compound. Yield: 2.5 gm

### Step b: Ethyl cyclopentyl (hydroxy)-2-thienylacetate

A solution of cyclopentyl bromide (2.91 g, 19.56 mmol) in tetrahydrofuran was added to a suspension of magnesium (0.46 g, 19.56 mmol) in tetrahydrofuran dropwise and stirred the reaction mixture at room temperature for 2 hour. The reaction mixture was cooled in an ice bath followed by the addition of a compound obtained from step a above (2.4 g, 13.043 mmol) diluted in tetrahydrofuran. The resulting reaction mixture was stirred for 1 hour in ice bath and then at room temperature for overnight. The reaction mixture was quenched with aqueous ammonium chloride and extracted with ethyl acetate. The organic layer was dried over anhydrous sodium sulphate and concentrated under reduced pressure. The residue thus obtained was purified by column chromatography to furnish the title compound. Yield: 640 mg.

### Step c: Cyclopentyl (hydroxy)-2-thienylacetic acid

A solution of a compound obtained from step b above (640 mg) in ethanol (20 ml) and potassium hydroxide (13.513 mmol) was stirred at room temperature for overnight. The reaction mixture was concentrated under reduced pressure. The residue thus obtained was diluted with water and extracted with dichloromethane. The aqueous layer was basified with sodium hydroxide and extracted with ethyl acetate. The organic layer was washed with brine, dried over anhydrous sodium sulphate and concentrated under reduced pressure. The residue thus obtained was purified by column chromatography using 20% ethyl acetate in hexane solvent mixture to furnish the little compound. Yield: 400 mg.

¹H NMR (CDCl₃): 7.24-7.23 (m, 1H), 7.16-7.15 (m, 1H), 7.00-6.90 (m, 1H), 3.3-2.82 (bs, 1H), 2.70-2.62 (m, 1H), 1.78-1.26 (m, 8H).

Analogs of cyclopentyl (hydroxy)-2-theinylacetic acid described below, were prepared similarly.
Hydroxy(phenyl)-2-thienyl acetic acid
Hydroxy(phenyl)-3-thienyl acetic acid
Hydroxy(di-2-thienyl)acetic acid
Hydroxy(4-methylphenyl)-2-thienyl acetic acid
(4-Fluorophenyl)(hydroxy)phenyl acetic acid
Hydroxy(4-methylphenyl)phenyl acetic acid

### Synthesis of hydroxy(di-4-methylphenyl)acetic acid and hydroxy(di-4-fluorophenyl)acetic acid

The title compound was prepared following the procedure described in Journal of American Chemical Society, 1953, 75, pg. 2654-2657.

### Synthesis of 2-cylobutyl-2-hydroxy-2-phenyl acetic acid, 2-cylopentyl-2-hydroxy-2-phenyl acetic acid, 2-cylohexyl-2-hydroxy-2-phenyl acetic acid and 2-cycloheptyl-2-hydroxy-2-2-phenyl acetic acid

The title compounds were prepared following the procedure described in Journal of Organic Chemistry, 2000, 65, pg. 6283-6287.

### Synthesis of 3-hydroxy-3,3-diphenyl propanoic acid

### Step a: Ethyl 3-hydroxy-3,3-diphenyl propanoate

To activated zinc dust (706 mg) was added a solution of benzophenone (2 g) and bromoethyl acetate (1 ml) in dry ether (5 ml) dropwise followed by the addition of a small crystal of iodine. The suspension was warmed to 40° C. for 3-4 hours followed by cooling it to 0° C. To it was added sulphuric acid (10%) and extracted with ethyl acetate. The combined organic layer was washed with sulphuric acid (10%) water, brine, dried and concentrated under reduced pressure. The residue thus obtained was purified by column chromatography using 5% ethyl acetate in hexane. Yield: 2.2 g.

### Step b: 3-Hydroxy-3,3-diphenylpropanoic acid

To a cold of solution of the compound obtained from step a above (2.1 g) in methanol, was added aqueous solution of potassium hydroxide and warmed the resulting reaction mixture to room temperature and then refluxed for 1.5 hours. The reaction mixture was concentrated under reduced pressure and the residue thus obtained was taken in water and washed with ethyl acetate. Aqueous layer was neutralized with hydrochloric acid and extracted with ethyl acetate. The organic layer was washed with water, brine, dried and concentrated under reduced pressure to furnish the title compound. Yield: 1.5 g.

¹H NMR (DMSO-d₆): 7.45 (m, 4H), 7.27 (m, 4H), 7.16 (m, 2H), 3.27 (s, 2H); IR (KBr): 3478, 1687.8 cm⁻¹; m.p: 217-220° C.

### Synthesis of hydroxy(2-hydroxy-5-methylphenyl)phenyl acetic acid

### Step a: Ethyl [2-(benzyloxy)-5-methyl phenyl](hydroxy)phenylacetate

A solution of 1-(benzyloxy)-2-bromo-4-methylbenzene (2 g) in dry tetrahydrofuran (2 ml) was cooled to −78° C. under nitrogen atmosphere followed by the addition of butyl lithium (6.73 ml) and stirred for 1 hour. To it was added a solution of ethyl oxo(phenyl)acetate (1.92 g) in dry tetrahydrofuran and stirred the reaction mixture for 1 hour at room temperature. The reaction mixture was quenched with aqueous ammonium chloride and extracted with ethyl acetate. The organic layer was washed with water and brine, dried over anhydrous sodium sulphate and evaporated under reduced pressure. The residue thus obtained was purified by column chromatography using 2% ethyl acetate in hexane solvent mixture to furnish the title compound. Yield: 2 g.

### Step b: [2-(Benzyloxy)-5-methylphenyl](hydroxy)phenyl acetic acid

A solution of the compound obtained from step a above (2 g) in ethanol (10 ml) and potassium hydroxide (1.5 g) was refluxed for 3 hours. The reaction mixture was concentrated under reduced pressure and the residue thus obtained was diluted with water and extracted with dichloromethane. The aqueous layer was basified with sodium hydroxide and extracted with ethyl acetate. The organic layer was washed with brine, dried over anhydrous sodium sulphate and concentrated under reduced pressure. The residue thus obtained was purified by column chromatography using 20% ethyl acetate in hexane solvent mixture to furnish the title compound. Yield: 500 mg.

### Step c: Hydroxy (2-hydroxy-5-methylphenyl)phenyl acetic acid

To a solution of the compound obtained from step b above (500 mg) in methanol (25 ml), was added palladium on carbon (100 g, 10%) under nitrogen atmosphere and anhydrous ammonium formate (450 mg) with constant stirring. The resulting reaction mixture was refluxed for 30 minutes followed by cooling it to room temperature. The reaction mixture was filtered through a bed of hyflo, washed with methanol, ethyl acetate and water. The filtrate was concentrated under reduced pressure. The residue thus obtained was diluted with water and pH of the resulting solution was adjusted to 14 with 1N sodium hydroxide. It was extracted with ether. The aqueous layer was acidified and extracted with ethylacetate. The organic layer was washed with water and brine, dried over anhydrous sodium sulphate and concentrated under reduced pressure to furnish the title compound. Yield: 320 mg.

¹H NMR δ: 7.37 (m, 6H), 7.21-7.06 (m, 2H), 3.5 (bs, 1H), 2.33 (s, 3H).

### Synthesis of 3-cyclohexyl-2-hydroxy-2-phenyl propanoic acid

### Step a: Ethyl 3-cyclohexyl-2-hydroxy-2-phenylpropanoate

To a solution of the compound ethyl mandalate (4 g) in tetrahydrofuran (20 ml), was added lithium diisopropyl amide (4 eq.) and stirred at −78° C. for 30 minutes. The reaction mixture was subsequently stirred at 0° C. for approximately 2 hours and then at room temperature for 1 hour. The reaction mixture was cooled to −78° C. and cyclohexylmethyl bromide (5.84 g) in tetrahydrofuran was added. The reaction mixture was stirred at the same temperature for 30 minutes and then at room temperature for overnight. The reaction mixture was quenched with aqueous ammonium chloride and extracted with ethyl acetate. The organic layer was dried over anhydrous sodium sulphate and concentrated under reduced pressure. The residue thus obtained was purified by column chromatography to furnish the title compound.

Yield=2.55 g.

### Step b: 3-Cyclohexyl-2-hydroxy-2-phenylpropanoic acid

To a solution of the compound obtained from step a above (0.93 g) in methanol (5 ml) was added aqueous potassium hydroxide (10 ml) and stirred at room temperature for overnight. The solvent was concentrated under reduced pressure and the residue thus obtained was diluted with water and extracted with dichloromethane. The aqueous layer was acidified with concentrated hydrochloric acid and extracted with ethyl acetate. The organic layer was dried and concentrated under reduced pressure to furnish the title compound. Yield=780 mg.

¹H NMR (CDCl₃): δ 7.26 (m, 2H), 7.30 (m, 3H), 2.15 (m, 1H), 1.98 (m, 1H), 1.76 (m, 1H), 1.61 (m, 2H), 1.25-0.93 (m, 5H); IR (KBr): 3435 and 1721 cm⁻¹; m.p.: 138-139.5° C.

The analog of 3-cyclohexyl-2-hydroxy-2-phenyl propanoic acid, described below was prepared similarly.

### 2-Hydroxy-2-phenylpent-4-ynoic acid

### Synthesis of 2-chloromethyl-5-methylpyridine hydrochloride

To 2-hydroxymethyl-5-methyl-pyridine (123 mg, 1 mmol) in chloroform at 0° C., thionyl chloride (2.5 mmol) was added slowly and stirred at 0° C. overnight. The solvent was evaporated under reduced pressure and the residue thus obtained was crystallized using hexane.

### Synthesis of 3-benzyl-2-methyl-3-aza-bicyclo[3.1.0]-hex-6-yl methanol and 3-benzyl-2-methyl-3-aza-bicyclo[3.1.0]-hex-6-yl methylamine

The title compound was prepared following the procedure as described in EP 0413455A2

### Synthesis of 3-benzy-3-azabicyclo[3.1.0]hex-6-yl-amine

The title compound was prepared following the procedure described in EP 0413455.

### Synthesis of 3-benzyl-3-azabicyclo[3.1.0]hex-6-yl-methyl amine

The title compound was prepared following the procedure described in EP 0413455, U.S. Pat. No. 2,490,714 and Synlett. 1097-1102 (1996).

### Synthesis of 3-benzyl-6-hydroxymethyl-3-azabicyclo[3.1.0]hexane

The title compound was prepared following the procedure described in EP 0413455.

### Synthesis of 2-hydroxy-2,2-diphenyl acetic acid

The title compound was prepared following the procedure described in Vogel's text book of practical organic chemistry page 1046 (5^{th} ED), J. Am. Chem. Soc. 75, 2654 (1953) and EP 613232.

### Synthesis of methoxy (4-methylphenyl) phenylacetic acid

### Step a: Methyl methoxy (4-methylphenyl)phenyl acetate

A solution of compound methyl hydroxy (4-methylphenyl)phenyl acetate (2.9 g) in dimethyl formamide (30 ml) was added dropwise to a suspension of sodium hydride (543 mg) in dimethylformamide (543 mg) at 0° C. and warmed the reaction mixture to room temperature. The reaction mixture was stirred for 1 hour, which was subsequently cooled to 0° C. followed by the addition of methyl iodide (0.85 ml). The reaction mixture was again stirred for 45 minutes at the 0° C. and then at room temperature for overnight. The reaction mixture was quenched with water and extracted with ethyl acetate. The organic layer was washed with water and brine, dried over anhydrous sodium sulphate and concentrated under reduced pressure. The residue thus obtained was purified by column chromatography using 10% ethyl acetate in hexane solvent mixture to furnish the title compound. Yield: 2.1 g.

### Step b: Methoxy(4-methyl phenyl)phenylacetic acid

Solution of the compound obtained from step a above (2.1 g) in methanol (50 ml) containing methanolic potassium hydroxide (40%, 13 ml) was refluxed for 3 hours and then stirred at room temperature for overnight. The reaction mixture was concentrated under reduced pressure and the residue thus obtained was diluted with water and washed with dichloromethane. The aqueous layer was acidified with concentrated hydrochloric acid and extracted with ethyl acetate. The organic layer was washed with water, dried and concentrated under reduced pressure. Yield: 1.5 g.

¹H NMR δ: 7.47-7.16 (m, 9H), 3.13 (s, 3H), 2.36 (s, 3H).

IR: 1713.3 cm⁻¹.

Analogs of methoxy (4-methylphenyl)phenylacetic acid described below, were prepared similarly,

Methoxy(diphenyl)acetic acid.

Propoxy(diphenyl)acetic acid.

### Synthesis of 3-benzyl-N-ethyl-3-azabicyclo[3.1.0]hexan-6-amine

To a solution of tetrahydrofuran at −78° C. was added lithium aluminium hydride (0.76 g) followed by the addition of a solution of 3-benzyl-N-acetyl-3-azabicyclo[3.1.0]hexan-6-amine (2.3 g) in tetrahydrofuran. The reaction mixture was warmed to room temperature and then refluxed for overnight. The reaction mixture was cooled in dry ice-acetone bath and quenched by addition of methanol followed by aqueous ammonium chloride solution. The reaction mixture was warmed to room temperature and washed with ethyl acetate. The organic layer was concentrated and purified with column chromatography using dichloromethane in triethyl amine (99:1) as eluent.

¹H NMR (CDCl₃): δ 7.19-7.31 (m, 5H), 3.5 (s, 2H), 2.95 (d, J=9 Hz, 2H), 2.65-2.73 (q, 2H), 2.47 (s, 1H), 2.39 (d, J=9 Hz, 2H), 1.29 (s, 2H), 1.06-1.11 (t, 3H).

### Scheme I

### EXAMPLE 1

### Synthesis of N-(3-benzyl-3-azabicyclo[3.1.0]hex-6-yl)-2-hydroxy-2-phenyl-2-(2-thienyl)acetamide (Compound No. 1)

A solution of hydroxy(phenyl)-2-thienylacetic acid (0.35 g, 1.49 mmol) and 3-benzyl-3-azabicyclo[3.1.0]hexan-6-amine (0.308 g, 1.639 mmol) in dimethylformamide (5 ml) was cooled in an ice bath followed by the addition of hydroxybenzotriazole (0.22 g, 1.039 mmol) and N-methylmorpholine (0.301 g, 2.98 mmol). The resulting reaction mixture was stirred in ice bath for 1 hour. 1-Ethyl-3-(3-dimethylaminopropyl) carbodiimide (0.284 g, 1.49 mmol) was added followed by stirring in ice bath for 1 hour and then at room temperature for overnight. The reaction mixture was poured into saturated sodium bicarbonate solution and extracted with ethyl acetate. The organic layer was washed with water, dried over anhydrous sodium sulphate and concentrated under reduced pressure. The residue thus obtained was purified by column chromatography using 30% ethylacetate in hexane solvent mixture as eluent. Yield=460 mg.

¹H NMR (CDCl₃): δ 7.43-7.20 (m, 1H), 6.95-6.94 (d, 2H), 6.24 (s, 1H), 4.1 (bs, 1H), 3.53 (s, 2H), 3.11-3.05 (m, 3H), 2.37-2.35 (m, 2H), 1.49 (m, 2H); IR (DCM): 1659.3 cm⁻¹; m.p.: 125-126.5° C.

Analogs of N-(3-benzyl-3-azabicyclo[3.1.0]hex-6-yl)-2-hydroxy-2-phenyl-2-(2-thienyl)acetamide (Compound No. 1) described below, were prepared similarly,

### N-[(3-Benzyl-3-azabicyclo[3.1.0]hex-6-yl)methyl]-2-hydroxy-2-phenyl-2-(2-thienyl)acetamide (Compound No. 2)

¹H NMR (CDCl₃): δ 7.51-7.48 (m, 2H), 7.34-7.23 (m, 9H), 7.00-6.95 (m, 2H), 6.35 (bs, 1H), 4.1 (bs, 1H), 3.57 (s, 2H), 3.19-3.14 (m, 2H), 2.94-2.87 (m, 2H), 2.34-2.31 (m, 2H), 1.47 (m, 1H), 1.32 (s, 2H).

### N-(3-Benzyl-3-azabicyclo[3.1.0]hex-6-yl)-2-hydroxy-2-phenyl-2-(3-thienyl)acetamide (Compound No. 5)

¹H NMR (CDCl₃): δ 7.41-6.95 (m, 13H), 6.31 (s, 1H), 3.8 (bs, 1H), 3.55 (s, 2H), 3.13-3.08 (m, 3H), 2.40-2.37 (d, 2H), 1.42 (m, 3H); IR (DCM): 1658 cm⁻¹.

### N-[(3-Benzyl-3-azabicyclo[3.1.0]hex-6-yl)methyl]-2-hydroxy-2-phenyl-2-(3-thienyl)acetamide (Compound No. 6)

¹H NMR (CDCl₃): δ 7.46-6.99 (m, 13H), 6.39 (bs, 1H), 4.01 (bs, 1H), 3.59 (s, 2H), 3.18-3.14 (m, 2H), 2.97-2.94 (m, 2H), 2.37-2.34 (m, 2H), 1.29-1.26 (m, 3H); IR (DCM): 1661 cm⁻¹.

### N-[(3-Benzyl-3-azabicyclo[3.1.0]hex-6-yl)methyl]-2-hydroxy-N-methyl-2-phenyl-2-(3-thienyl)acetamide (Compound No. 8)

¹H NMR (CDCl₃): δ 7.408-7.14 (m, 13H), 6.2 (bs, 1H), 3.59 (s, 2H), 3.40 (m, 2H), 3.1 (m, 2H), 2.94 (m, 2H), 2.59 (s, 3H), 1.28 (m, 3H); IR (DCM): 1631 cm⁻¹.

### N-[(3-Benzyl-3-azabicyclo[3.1.0]hex-6-yl)methyl]-2-hydroxy-2,2-di-2-thienylacetamide (Compound No. 10)

¹H NMR (CDCl₃): δ 7.32-7.17 (m, 6H), 6.97-6.8 (m, 5H), 6.01 (bs, 1H), 4.8 (bs, 1H), 3.59 (s, 2H), 3.19-3.11 (m, 2H), 2.97-2.87 (m, 2H), 2.37-2.34 (m, 2H), 1.302-1.26 (m, 3H); IR (DCM): 1663 cm⁻¹.

### N-[(3-Benzyl-3-azabicyclo[3.1.0]hex-6-yl)methyl]-2-hydroxy-N-methyl-2-phenyl-2-(2-thienyl)acetamide (Compound No. 11)

¹H NMR (CDCl₃): δ 7.44-7.28 (m, 10H), 6.97-6.96 (m, 3H), 3.59 (s, 2H), 3.39 (m, 2H), 3.1 (m, 2H), 2.95 (m, 2H), 2.66 (s, 3H), 1.41 (m, 3H); IR (DCM): 1634 cm⁻¹.

### N-[(3-Benzyl-3-azabicyclo[3.1.0]hex-6-yl)methyl]-2-cyclopentyl-2-hydroxy-N-methyl-2-(2-thienyl)acetamide (Compound No. 12)

¹H NMR (CDCl₃): δ 7.28-7.21 (m, 6H), 7.015-7.007 (m, 1H), 6.93-6.90 (m, 1H), 5.76 (s, 1H), 3.56 (s, 2H), 3.42-2.90 (m, 8H), 2.32 (m, 2H), 1.65-0.88 (m, 11H); IR (DCM): 1625 cm⁻¹.

### N-[(3-Benzyl-3-azabicyclo[3.1.0]hex-6-yl)methyl]-2-hydroxy-N-methyl-2-(4-methylphenyl)-2-(2-thienyl)acetamide (Compound No. 13)

¹H NMR (CDCl₃): δ 7.35-7.13 (m, 10H), 6.97-6.95 (m, 2H), 6.17 (bs, 1H), 3.60-2.67 (m, 11H), 2.34 (m, 5H), 1.23 (m, 3H); IR (DCM): 1633 cm⁻¹.

### N-[(3-Benzyl-3-azabicyclo[3.1.0]hex-6-yl)methyl]-2-hydroxy-2-(4-methylphenyl)-2-(2-thienyl)acetamide (Compound No. 16)

¹H NMR (CDCl₃): δ 7.38-6.94 (m, 12H), 6.37 (bs, 1H), 3.59 (s, 2H), 3.18-3.14 (m, 1H), 2.97-2.87 (m, 3H), 2.36-2.33 (m, 5H), 1.30-1.25 (m, 3H).

### N-(3-Benzyl-3-azabicyclo[3.1.0]hex-6-yl)-2-(4-fluorophenyl)-2-hydroxy-2-phenylacetamide (Compound No. 20)

¹H NMR (CDCl₃): δ 7.40-7.20 (m, 12H), 6.98 (m, 2H), 6.38 (bs, 1H), 3.55 (s, 2H), 3.13 (bs, 1H), 3.10 (m, 2H), 2.38 (m, 3H), 1.48 (m, 2H); m/z: 417 (M⁺+1).

### N-(3-Benzyl-3-azabicyclo[3.1.0]hex-6-yl)-2-hydroxy-2-(4-methylphenyl)-2-phenylacetamide (Compound No. 21)

¹H NMR (CDCl₃): δ 7.40-7.12 (m, 14H), 6.31 (bs, 1H), 3.79 (bs, 1H), 3.57 (s, 2H), 3.11 (m, 3H), 2.41 (m, 2H), 2.33 (s, 3H), 1.50 (m, 2H); m/z: 413 (M⁺+1).

### N-[(3-Benzyl-3-azabicyclo[3.1.0]hex-6-yl)methyl]-3-hydroxy-N-methyl-3,3-diphenylpropanamide (Compound No. 23)

¹H NMR (CDCl₃): δ 7.41-7.15 (m, 15H), 6.78 (bs, 1H), 3.57 (m, 2H), 3.20 (m, 4H), 3.00 (m, 3H), 2.88 (m, 2H), 2.37-2.26 (m, 2H), 1.33-1.27 (m, 2H), 1.15 (m, 1H); IR: 3331 and 1619 cm⁻¹; m/z: 441.5 (M⁺+1).

### N-[(3-Benzyl-3-azabicyclo[3.1.0]hex-6-yl)methyl]-3-hydroxy-3,3-diphenylpropanamide (Compound No. 24)

¹H NMR (CDCl₃): δ 7.42-7.15 (m, 15H), 5.8 (bs, 1H), 3.59 (s, 2H), 3.08 (s, 2H), 2.95 (m, 4H), 2.32 (m, 2H); IR: 3320 and 1666 cm⁻¹; m/z: 427.4 (M⁺+1).

### N-[(3-Benzyl-3-azabicyclo[3.1.0]hex-6-yl)methyl]-2-hydroxy-2-(2-hydroxy-5-methylphenyl)-2-phenylacetamide (Compound No. 26)

¹H NMR (CDCl₃): δ 7.30 (m, 11H), 7.05 (m, 1H), 6.94 (m, 1H), 6.82 (m, 1H), 3.59 (s, 2H), 3.3-3.12 (m, 2H), 2.96 (m, 2H), 2.37 (m, 2H), 2.26 (s, 3H), 1.49 (m, 1H), 1.33 (m, 2H); m/z: 443.3 (M⁺+1); IR: 3400 and 1648 cm⁻¹.

### N-[(3-Benzyl-3-azabicyclo[3.1.0]hex-6-yl)methyl]-2-hydroxy-2,2-bis(3-methylphenyl)acetamide (Compound No. 29)

¹H NMR (CDCl₃): δ 7.21 (m, 13H), 6.32 (bs, 1H), 4.0 (bs, 1H), 3.57 (s, 2H), 3.16 (m, 2H), 2.93 (m, 2H), 2.32 (m, 8H), 1.46 (m, 1H), 1.28 (m, 2H); IR: 3403 and 1658.8 cm⁻¹; m/z: 441 (M⁺+1).

### N-[(3-Benzyl-3-azabicyclo[3.1.0]hex-6-yl)methyl]-2-methoxy-2,2-diphenylacetamide (Compound No. 32)

¹H NMR (CDCl₃): δ 7.43 (m, 3H), 7.28 (m, 12H), 3.55 (s, 2H), 3.10 (m, 2H), 3.00 (s, 3H), 2.93 (m, 2H), 2.31 (m, 2H), 1.43 (m, 1H), 1.31 (m, 2H); IR: 3424 and 1674 cm⁻¹; m/z: 427.4 (M⁺+1).

### N-(3-Benzyl-3-azabicyclo[3.1.0]hex-6-yl)-2-methoxy-2,2-diphenylacetamide (Compound No. 33)

¹H NMR (CDCl₃): δ 7.44-7.24 (m, 15H), 3.55 (s, 2H), 3.08 (m, 3H), 3.00 (s, 3H), 2.38 (m, 2H), 1.52 (m, 2H), 1.29 (m, 1H); m/z: 413.4 (M⁺+1); IR (KBr): 3338 and 1660 cm⁻¹.

### N-[(3-Benzyl-3-azabicyclo[3.1.0]hex-6-yl)methyl]-2-methoxy-N-methyl-2,2-diphenylacetamide (Compound No. 34)

¹H NMR (CDCl₃): δ 7.49 (m, 5H), 7.20 (m, 10H), 3.57 (s, 2H), 3.34 (m, 5H), 2.92 (m, 2H), 2.85 (s, 3H), 2.33 (m, 2H), 1.41 (m, 2H), 0.88 (m, 1H); IR: 1641 cm⁻¹; m/z: 441.5 (M⁺+1).

### N-(3-benzyl-3-azabicyclo[3.1.0]hex-6-yl)-N-ethyl-2-hydroxy-2,2-diphenylacetamide (Compound No. 49)

¹H NMR (CDCl₃): δ 7.31 (m, 15H), 6.09 (bs, 1H), 3.50 (m, 2H), 3.06 (m, 5H), 2.40 (m, 2H), 1.60 (m, 4H), 1.30 (m, 1H), 0.88 (m, 1H); IR: 3358 and 1628 cm⁻¹; m/z: 427 (M⁺+1).

### (3-Benzyl-2-methyl-3-azabicyclo[3.1.0]hex-6-yl)methyl cyclohexyl(hydroxy)phenylacetate (Compound No. 59)

¹HNMR (CDCl₃): δ 7.65 (m, 2H), 7.29 (m, 8H), 4.2-3.86 (m, 3H), 3.71 (bs, 1H), 3.13 (m, 1H), 2.84 (m, 1H), 2.68 (m, 1H), 2.28 (m, 2H), 1.74-1.05 (m, 16H); MS: 434 (M⁺+1). IR: 3509 and 1720 cm⁻¹.

### N-[(3-Benzyl-2-methyl-3-azabicyclo[3.1.0]hex-6-yl)methyl]-2-cyclopentyl-2-hydroxy-2-phenylacetamide (Compound No. 60)

¹HNMR (CDCl₃): δ 7.60 (m, 2H), 7.30 (m, 8H), 6.6 (bs, 1H), 3.94 (m, 1H), 3.5-3.2 (m, 2H), 3.15-2.8 (m, 5H), 2.5 (m, 1H), 1.75-1.1 (m, 14H); IR (KBr): 3410 and 1655 cm⁻¹.

### N-[(3-Benzyl-2-methyl-3-azabicyclo[3.1.0]hex-6-yl)methyl]-2-cyclohexyl-2-hydroxy-2-phenylacetamide (Compound No. 61)

¹HNMR (CDCl₃): δ 7.59 (m, 2H), 7.46 (m, 4H), 7.29 (m, 4H), 7.08 (bs, 1H), 4.10 (m, 2H), 3.50 (m, 2H), 3.07 (m, 3H), 2.39 (m, 1H), 2.2 (m, 1H), 2.0-1.75 (m, 7H), 1.45-1.1 (m, 8H), 0.88 (m, 1H); IR (KBr): 3407 and 1656 cm⁻¹.

### N-[(3-Benzyl-3-azabicyclo[3.1.0]hex-6-yl)methyl]-2-cycloheptyl-2-hydroxy-2-phenylacetamide (Compound No. 77)

¹HNMR (CDCl₃): δ 7.59 (m, 2H), 7.30 (m, 8H), 6.65 (bs, 1H), 3.58 (s, 2H), 3.10-2.91 (m, 6H), 2.62 (m, 1H), 2.33 (m, 2H), 1.69-1.10 (m, 11H); MS: 432 (M⁺+1); IR: 3413 and 1654 cm⁻¹.

### N-[(3-Benzyl-3-azabicyclo[3.1.0]hex-6-yl)methyl]-2-cyclohexyl-2-hydroxy-N-methyl-2-phenylacetamide (Compound No. 78)

¹HNMR (CDCl₃): δ 7.28 (m, 10H), 4.02 (m, 1H), 3.51-2.77 (m, 9H), 2.41 (m, 2H), 1.82-1.1 (m, 13H); MS: 433 (M⁺+1); IR: 3422 and 1621 cm⁻¹.

### N-[(3-Benzyl-3-azabicyclo[3.1.0]hex-6-yl)methyl]-2-hydroxy-N-methyl-2,2-diphenylacetamide (Compound No. 79)

¹HNMR (CDCl₃): δ 7.30 (m, 15H), 5.90 (bs, 1H), 3.7-3.6 (m, 3H), 3.13-2.6 (m, 4H), 2.5 (m, 4H), 1.6-0.8 (m, 3H); MS: 427 (M⁺+1); IR: 3355 and 1630 cm⁻¹.

### N-[(3-Benzyl-3-azabicyclo[3.1.0]hex-6-yl)methyl]-2-hydroxy-3-methyl-2-phenylpentanamide (Compound No. 85)

¹HNMR (CDCl₃): δ 7.60 (m, 2H), 7.30 (m, 8H), 6.66 (bs, 1H), 3.59 (s, 2H), 3.09-2.91 (m, 4H), 2.48 (m, 1H), 2.34 (m, 2H), 2.0 (bs, 1H), 1.48 (m, 1H), 1.38 (m, 1H), 1.25 (m, 2H), 0.97 (m, 3H), 0.87 (m, 2H), 0.71 (m, 2H); MS: 393 (M⁺+1); IR: 3407 and 1654 cm⁻¹.

### N-[(3-Benzyl-3-azabicyclo[3.1.0]hex-6-yl)methyl]-2-cyclopentyl-2-methoxy-2-phenylacetamide (Compound No. 86)

¹HNMR (CDCl₃): δ 7.46 (m, 2H), 7.27 (m, 8H), 6.92 (bs, 1H), 3.57 (s, 2H), 3.20-3.08 (m, 5H), 2.93 (m, 3H), 2.34 (m, 2H), 1.88-1.66 (m, 2H), 1.50 (m, 6H), 1.2 (m, 3H); MS: 419 (M⁺+1); IR: 3425 and 1671 cm⁻¹.

### N-[(3-Benzyl-3-azabicyclo[3.1.0]hex-6-yl)methyl]-3-cyclohexyl-2-hydroxy-2-phenylpropanamide (Compound No. 91)

¹HNMR (CDCl₃): δ 7.57 (m, 2H), 7.33 (m, 8H), 6.62 (bs, 1H), 3.61 (m, 2H), 3.08-2.97 (m, 5H), 2.38 (m, 2H), 2.20 (m, 1H), 1.97 (m, 1H), 1.77-1.4 (m, 7H), 1.27-0.98 (m, 7H).

MS: 433 (M⁺+1); IR (KBr): 3409 and 1644 cm⁻¹.

### N-(3-Benzyl-3-azabicyclo[3.1.0]hex-6-yl)-2-cyclohexyl-N-ethyl-2-hydroxy-2-phenylacetamide (Compound No. 93)

¹HNMR (CDCl₃): δ 7.62-6.81 (M, 10h), 3.63 (S, 2h), 3.37-2.44 (M, 9h), 1.78-1.05 (M, 15H).

### N-[(3-benzyl-3-azabicyclo[3.1.0]hex-6-yl)methyl]-3,33-trifluoro-2-hydroxy-2-phenylpropanamide (Compound No. 96)

¹HNMR (CDCl₃): δ 7.63 (m, 2H), 7.40 (m, 3H), 7.27 (m, 5H), 3.58 (s, 2H), 3.16 (m, 2H), 2.93 (m, 2H), 2.35 (m, 2H), 1.42 (m, 1H), 0.85 (m, 2H); MS: 405 (M⁺+1); IR: 3417 and 1675 cm⁻¹.

### N-[(3-benzyl-3-azabicyclo[3.1.0]hex-6-yl)methyl]-2-hydroxy-2-phenylpent-4-ynamide (Compound No. 98)

¹HNMR (CDCl₃): δ 7.63 (m, 2H), 7.38-7.23 (m, 8H), 6.22 (bs, 1H), 3.56 (s, 2H), 3.10 (m, 2H), 2.9 (m, 2H), 2.31 (m, 2H), 1.97 (s, 2H), 1.38 (m, 1H), 1.22 (m, 2H); MS: 375 (M⁺+1); IR: 3401 and 1664 cm⁻¹.

### N-[(3-benzyl-3-azabicyclo[3.1.0]hex-6-yl)methyl]-2-hydroxy-3-methyl-2-phenylbutanamide (Compound No. 101)

¹HNMR (CDCl₃): δ 7.62-0.59 (m, 2H), 7.35-7.21 (m, 8H), 6.87 (bs, 1H), 3.89-3.86 (m, 2H).

### N-[(3-Benzyl-3-azabicyclo[3.1.0]hex-6-yl)methyl]-2-hydroxy-N-methyl-2-phenyl-2-pyridin-3-ylacetamide (Compound No. 105)

¹H NMR (CDCl₃): δ 8.63-8.57 (2H, bd), 7.76 (1H, bs), 7.40-7.21 (11H, bs), 3.82 (2H, bm), 3.47 (2H, bm), 3.14 (2H, bs), 3.00 (1H, bs), 2.54 (3H, bs), 1.33 (2H, s), 0.88 (1H, bs); MS: 428 (M⁺+1).

### N-[(3-Benzyl-3-azabicyclo[3.1.0]hex-6-yl)methyl]-2-hydroxy-N-methyl-2-phenylhex-4-enamide (Compound No. 110)

¹H NMR (CDCl₃): δ 7.31 (10H, bs), 5.61 (1H, m), 5.47 (1H, bm), 3.86-2.51 (15H, m), 1.68 (4H, bs); MS: 405 (M⁺+1).

### N-[(3-Benzyl-3-azabicyclo[3.1.0]hex-6-yl)methyl]-2-hydroxy-2-phenyl-2-pyridin-3-ylacetamide (Compound No. 111)

¹H NMR (CDCl₃): δ 8.70 (1H, s), 8.51 (1H, bs), 7.80 (1H, d, 4 Hz), 7.38-7.23 (11H, m), 6.70 (1H, bs), 3.57 (2H, bs), 3.15 (2H, m), 2.95 (2H, bs), 2.36 (2H, bs), 1.31 (2H, m), 0.88 (1H, bs); MS: 414 (M⁺+1).

### N-[(3-Benzyl-3-azabicyclo[3.1.0]hex-6-yl)methyl]-2-hydroxy-2-phenylhex-4-enamide (Compound No. 112)

¹H NMR (CDCl₃): δ 7.62 (2H, d, 8 Hz), 7.35-7.24 (8H, m), 6.81 (1H, bs), 5.66 (1H, m), 5.30 (1H, m), 3.60 (2H, s), 3.13-3.11 (1H, m), 3.03-2.99 (4H, m), 2.37 (1H, m), 2.2 (2H, bs), 1.68 (3H, d, 8 Hz), 1.30-1.25 (3H, m); MS: 391 (M⁺+1).

### N-[(3-Benzyl-3-azabicyclo[3.1.0]hex-6-yl)methyl]-N-methyl-2,2-diphenyl-2-propoxyacetamide (Compound No. 113)

¹H NMR (CDCl₃): δ 7.50-7.48 (8H, bs), 7.33-7.15 (7H, m), 3.82 (2H, bs), 3.42-2.05 (10H, m), 1.61 (2H, m), 1.26 (2H, s), 1.01 (2H, t, 8 Hz), 0.86 (3H, t, 8 Hz).

MS: 469 (M⁺+1).

### N-[(3-Benzyl-3-azabicyclo[3.1.0]hex-6-yl)methyl]-N-methyl-2,2-diphenylpropanamide (Compound No. 119)

¹H NMR (CDCl₃): δ 7.35-7.28 (m, 15H, m), 3.65 (bs, 1H), 3.49 (bs, 1H), 3.32 (bs, 1H), 3.02-3.00 (m, 2H), 2.66 (bs, 1H), 2.34-2.22 (m, 5H), 1.88 (bs, 3H), 1.03 (m, 3H); MS: 425 (M⁺+1).

### EXAMPLE 1a

### Synthesis of (3-benzyl-3-azabicyclo[3.1.0]hex-6-yl)methyl hydroxy(phenyl)pyridin-2-ylacetate (Compound No. 117)

To the compound 3-benzyl-6-hydroxymethyl-3-azabicyclo[3.1.0]hexane (0.197 g, 1 eqi.) was added butyl lithium (0.062 g, 0.6 ml, 1 eqi.) at −78° C. and stirred the mixture for 30 minutes at the same temperature. To the resulting reaction mixture was added a solution of ethyl phenyl (hydroxy)-2-pyridylacetate (0.25 g, 1 eqi.) in tetrahydrofuran (10 ml). The mixture was allowed to warm at room temperature and subsequently stirred at room temperature for overnight. The mixture was quenched with aqueous ammonium chloride and extracted with ethyl acetate. The organic layer was washed with brine, dried over anhydrous sodium sulphate and concentrated under reduced pressure using 40% ethyl acetate in hexane solvent mixture as eluent to furnish the title compound. Yield: 60 mg.

¹H NMR (CDCl₃): δ 8.55 (1H, m), 7.55 (1H, m), 7.42 (2H, d, 8 Hz), 7.31 (1H, d, 8 Hz), 7.33-7.23 (9H, m), 4.11-4.06 (2H, m), 3.56 (2H, s), 2.90 (2H, d, 8 Hz), 2.7-2.8 (1H, m), 2.30 (2H, bd), 1.28 (3H, m); MS: 415 (M⁺+1).

### EXAMPLE 1b

### Synthesis of N-[(3-Benzyl-3-azabicyclo[3.1.0]hex-6-yl)methyl]-2-hydroxy-2-phenyl-2-pyridin-2-ylacetamide (Compound No. 1)

Diisobutyl aluminium (0.798 g, 5.612 mmol) was added to a solution of 3-benzyl-3-azabicyclo[3.1.0]hex-6-yl-methyl amine (1.179 g, 5.836 mmol) in tetrahydrofuran (2 ml) precooled to 0° C. The reaction mixture was allowed to warm to room temperature and stirred at the same for 2 hrs. The resulting reaction mixture was added to a solution of ethyl phenyl (hydroxy)-2-pyridylacetate in tetrahydrofuran (2 ml) under nitrogen at room temperature. The reaction mixture was quenched with aqueous ammonium chloride solution and extracted with ethyl acetate. The organic layer was washed with brine, dried and concentrated under reduced pressure and residue purified by column chromatography. Yield: 120 mg.

¹H NMR (CDCl₃): δ 7.8 (1H, bs), 7.71 (1H, m), 7.50 (2H, d=8 Hz), 7.28 (8H, bs), 6.96 (1H, bs), 3.56 (2H, s), 3.18-3.11 (2H, m), 2.94 (2H, t), 2.33 (2H, bs), 1.46 (1H, bs), 1.28 (2H, bs); MS: 414 (M⁺+1).

### EXAMPLE 2

### Synthesis of (3-benzyl-3-azabicyclo[3.1.0]hex-6-yl)methyl hydroxy(phenyl)-2-thienylacetate (Compound No. 3)

A solution of the compound hydroxy(phenyl)-2-thienylacetic acid (0.299 g), 3-benzyl-3-azabicyclo[3.1.0]hex-6-ylmethyl methane sulfonate (0.3 g, 1.067 mmol) and 1,8-diazabicyclo[5.4.0]undec-7-ene (0.32 g, 2.134 mmol) in toluene (15 ml) was refluxed for 6 hours and then stirred at room temperature for overnight. The reaction mixture was concentrated and the residue thus obtained was purified by column chromatography using 15% ethyl acetate in hexane solvent mixture to furnish the title compound. Yield=280 mg.

¹H NMR (CDCl₃): δ 7.52-7.49 (m, 2H), 7.32-7.25 (m, 9H), 7.13-7.12 (m, 1H), 6.98-6.95 (m, 1H), 4.86 (s, 1H), 4.14-4.10 (m, 2H), 3.68 (s, 2H), 3.01 (m, 2H), 2.52 (m, 2H), 1.29-1.25 (m, 3H); IR (DCM): 1727 cm⁻¹; m.p: 81.9-82.2° C. Analogs of (3-benzyl-3-azabicyclo[3.1.0]hex-6-yl)methyl hydroxy(phenyl)-2-thienylacetate (Compound No. 3) described below, were prepared similarly.

### (3-Benzyl-3-azabicyclo[3.1.0]hex-6-yl)methyl hydroxy(4-methylphenyl)-2-thienylacetate (Compound No. 14)

¹H NMR (CDCl₃): δ 7.40-6.94 (m, 12H), 4.45 (s, 1H), 4.13-4.09 (m, 2H), 3.57 (s, 2H), 2.93-2.91 (m, 2H), 2.32 (m, 5H), 1.66 (m, 1H), 1.33-1.30 (m, 2H).

### (3-Benzyl-3-azabicyclo[3.1.0]hex-6-yl)methyl hydroxy(di-2-thienyl)acetate (Compound No. 15)

¹H NMR (CDCl₃): δ 7.29-7.18 (m, 9H), 6.96-6.93 (m, 2H), 4.14-4.12 (m, 2H), 3.57 (s, 2H), 2.94-2.91 (d, 2H), 2.38-2.31 (m, 2H), 1.68 (m, 1H), 1.34 (m, 2H).

### (3-Benzyl-3-azabicyclo[3.1.0]hex-6-yl)methyl 3-hydroxy-3,3-diphenylpropanoate (Compound No. 22)

¹H NMR (CDCl₃): δ 7.43-7.16 (m, 15H), 5.06 (bs, 1H), 3.86 (d, J=9 Hz, 2H), 3.60 (m, 2H), 3.27 (s, 2H), 2.93 (m, 2H), 2.33 (m, 2H), 1.5 (m, 1H), 1.20 (m, 2H); IR (KBr): 3506 and 1707 cm⁻¹; MS: 428.4 (M⁺+1).

### (3-Benzyl-2-methyl-3-azabicyclo[3.1.0]hex-6-yl)methyl cyclopentyl(hydroxy)phenylacetate (Compound No. 58)

¹HNMR (CDCl₃): δ 7.67 (m, 2H), 7.27 (m, 8H), 4.02-3.85 (m, 3H), 3.75 (bs, 1H), 3.14 (m, 1H), 2.87 (m, 2H), 2.69 (m, 1H), 2.29 (m, 1H), 1.61-1.22 (m, 11H), 1.07 (m, 3H); IR: 3510 and 1719 cm⁻¹; MS: 420 (M⁺+1).

### (3-Benzyl-2-methyl-3-azabicyclo[3.1.0]hex-6-yl)methyl hydroxy(diphenyl)acetate (Compound No. 62)

¹HNMR (CDCl₃): δ 7.44 (m, 4H), 7.30 (m, 11H), 4.28 (bs, 1H), 4.17 (m, 1H), 4.0 (m, 1H), 3.88 (m, 1H), 3.11 (m, 1H), 2.84 (m, 1H), 2.65 (m, 1H), 2.27 (m, 1H), 1.35 (m, 1H), 1.22 (m, 2H), 1.04 (m, 3H); MS: 428 (M⁺+1); IR: 3492 and 1723 cm⁻¹.

### (3-Benzyl-3-azabicyclo[3.1.0]hex-6-yl)methyl 2-hydroxy-2-phenylpent-4-ynoate (Compound No. 97)

¹HNMR (CDCl₃): δ 7.55-7.2 (m, 10H), 3.88 (m, 2H), 3.57 (s, 2H), 2.98 (m, 2H), 2.35 (m, 2H), 2.05 (m, 2H), 1.91 (m, 1H), 1.28 (m, 3H); IR: 1738 cm⁻¹.

### (3-Benzyl-3-azabicyclo[3.1.0]hex-6-yl)methyl (4E or 4Z)-2-hydroxy-2-phenylhex-4-enoate (Compound No. 115)

¹H NMR (CDCl₃): δ 7.62 (2H, d, 8 Hz), 7.35-7.23 (8H, m), 5.60 (1H, m), 5.44 (1H, m), 4.06 (1H, m), 3.94 (1H, m), 3.69 (1H, s), 3.57 (2H, s), 2.95-2.91 (3H, m), 2.6 (1H, m), 2.32 (2H, bs), 1.64 (3H, d, 6 Hz), 1.32 (3H, bs).

### (3-Benzyl-3-azabicyclo[3.1.0]hex-6-yl)methyl (2R or 2S)-hydroxy(4-methylphenyl)phenylacetate (Compound No. 116)

¹H NMR (CDCl₃): δ 7.44-6.96 (14H, m), 6.34 (1H, bs), 3.58 (2H, bs), 3.15 (2H, bm), 2.95 (2H, m), 2.34-2.22 (5H, bs), 1.27 (3H, bs); HPLC: (96.43% pure); MS: 428 (M⁺+1).

### (3-Benzyl-3-azabicyclo[3.1.0]hex-6-yl)methyl hydroxy(phenyl)pyridin-3-ylacetate (Compound No. 118)

¹H NMR (CDCl₃): δ 8.72 (1H, s), 8.54 (1H, d, 4 Hz), 7.8 (1H, d, 8 Hz), 7.43 (2H, bs), 7.34-7.23 (9H, m), 4.33 (1H, bs), 4.18-4.07 (2H, m), 3.57 (2H, s), 2.91 (2H, d, 8 Hz), 2.31 (2H, d, 8 Hz), 1.32 (3H, m); MS: 415 (M⁺+1).

### Tert-butyl 6-({[(4E)-2-hydroxy-2-phenylhex-4-enoyl]oxy}methyl)-3-azabicyclo[3.1.0]hexane-3-carboxylate (Compound No. 120)

¹H NMR (CDCl₃): δ 7.62-7.60 (d, 2H), 7.38-7.30 (m, 3H), 5.62 (m, 1H), 5.50 (m, 1H), 4.07-4.08 (m, 2H), 3.68 (s, 1H), 3.54 (m, 1H), 3.43 (m, 1H), 3.31 (m, 2H), 2.95 (m, 1H), 2.70 (m, 1H), 1.68 (m, 3H), 1.4 (s, 9H).

### EXAMPLE 3

### Synthesis of N-(3-azabicyclo[3.1.0]hex-6-ylmethyl)-2-hydroxy-2,2-bis(3-methylphenyl)acetamide (Compound No. 28)

To a solution of the Compound No. 29 (0.21 g) in methanol (25.0 ml), was added palladium on carbon (50 mg, 10%) and anhydrous ammonium formate (0.15 g) with constant stirring. The resulting reaction mixture was refluxed for half an hour followed by cooling it to room temperature. The reaction mixture was filtered through a bed of hyflo, washed with methanol, ethylacetate and water. The filtrate was concentrated under reduced pressure. The residue thus obtained was diluted with water and pH of the resulting solution was adjusted to pH˜14 with 1N sodium hydroxide. The solution was extracted with ethyl acetate and the ethyl acetate layer was washed with water and brine, dried over anhydrous sodium sulphate and concentrated under reduced pressure to furnish the title compound. Yield: 84 mg.

¹H NMR (CDCl₃): δ 7.20 (m, 8H), 6.6 (bs, 1H), 3.3-3.03 (m, 6H), 2.40 (s, 6H), 1.5 (m, 1H), 0.92 (m, 2H); m/z: 351 (M⁺+1).

Analogs of N-(3-azabicyclo[3.1.0]hex-6-ylmethyl)-2-hydroxy-2,2-bis(3-methylphenyl)acetamide (Compound No. 28) described below, were prepared by deprotecting appropriate benzylated compound, respectively, as applicable in each case.

### N-(3-Azabicyclo[3.1.0]hex-6-ylmethyl)-2-hydroxy-2-phenyl-2-(2-thienyl)acetamide (Compound No. 18)

¹H NMR (CDCl₃): δ 7.51-7.48 (m, 2H), 7.36-7.30 (m, 4H), 7.02-6.96 (m, 2H), 6.61 (bs, 1H), 3.23-3.12 (m, 2H), 2.93-2.75 (m, 5H), 1.28 (s, 2H), 0.79 (m, 1H); m.p: 70-75° C.

### 3-Azabicyclo[3.1.0]hex-6-ylmethyl hydroxy[bis(3-methylphenyl)]acetate (Compound No. 27)

¹H NMR (CDCl₃): δ 7.27-7.13 (m, 8H), 4.17 (d, J=7.2 Hz, 2H), 2.85 (m, 4H), 2.33 (s, 6H), 1.37 (m, 2H), 0.97 (m, 1H); m/z: 352.3 (M⁺+1); m.p.: 105-108° C.

### N-(3-Azabicyclo[3.1.0]hex-6-ylmethyl)-2-hydroxy-N-methyl-2,2-bis(3-methylphenyl)acetamide (Compound No. 30)

¹H NMR (CDCl₃): δ 7.20 (m, 8H), 3.48 (m, 1H), 3.14 (m, 1H), 2.95 (m, 3H), 2.59 (m, 4H), 2.36 (s, 6H); m/z: 365 (M⁺+1).

### N-(3-Azabicyclo[3.1.0]hex-6-ylmethyl)-3-hydroxy-3,3-diphenylpropanamide (Compound No. 36)

¹H NMR (CDCl₃): δ 7.44-7.18 (m, 10H), 3.15 (s, 2H), 2.98 (m, 2H), 2.86 (m, 2H), 2.70 (m, 2H), 1.15 (m, 2H), 0.65 (m, 1H); IR (KBr): 3337.9 and 1640.2 cm⁻¹; m/z: 337.2 (M⁺+1).

### N-(3-azabicyclo[3.1.0]hex-6-ylmethyl)-2-methoxy-N-methyl-2,2-diphenylacetamide (Compound No. 38)

¹H NMR (CDCl₃): δ 7.57 (m, 4H), 7.44-7.28 (m, 6H), 3.58-3.38 (m, 4H), 3.12-2.84 (m, 6H), 2.21 (m, 2H), 1.61 (m, 2H), 1.09 (m, 1H); IR: 3421 and 1639.4 cm⁻¹; m/z: 351.2 (M⁺+1).

### N-3-azabicyclo[3.1.0]hex-6-yl-2-methoxy-2,2-diphenylacetamide (Compound No. 39)

¹H NMR (CDCl₃): δ 7.42-7.18 (m, 10H), 3.18 (m, 2H), 3.05 (m, 5H), 2.54 (5.1H), 2.08 (m, 2H), 1.28 (m, 2H); m/z: 323.2 (M⁺+1); IR: 3409 and 1666 cm⁻¹.

### N-(3-Azabicyclo[3.1.0]hex-6-ylmethyl)-2-methoxy-2,2-diphenylacetamide (Compound No. 40)

¹H NMR (CDCl₃): δ 7.46-7.32 (m, 10H), 3.20 (m, 2H), 3.04 (s, 3H), 2.96 (m, 2H), 2.86 (m, 2H), 1.28 (m, 2H), 0.83 (m, 1H); m/z: 337.2 (M⁺+1); IR: 3419 and 1664.6 cm⁻¹.

### N-(3-Azabicyclo[3.1.0]hex-6-ylmethyl)-2-methoxy-2-(4-methylphenyl)-2-phenylacetamide (Compound No. 41)

¹H NMR (CDCl₃): δ 7.38 (m, 2H), 7.24 (m, 5H), 7.09 (m, 2H), 3.10 (m, 2H), 2.90 (m, 6H), 2.28 (m, 4H), 1.23 (m, 2H), 0.80 (m, 1H); IR (KBr): 3423 & 1667 cm⁻¹.

### 3-Azabicyclo[3.1.0]hex-6-ylmethyl diphenyl(propoxy)acetate (Compound No. 42)

¹H NMR (CDCl₃): δ 7.46 (m, 4H), 7.30 (m, 6H), 4.02 (m, 2H), 3.20 (m, 2H), 2.82 (m, 2H), 2.28 (m, 2H), 1.60 (m, 2H), 1.40 (m, 1H), 1.27 (m, 2H), 0.90 (t, J=7.2 Hz, 3H). IR: 3423 and 1739 cm⁻¹; m/z: 366 (M⁺+1).

### N-(3-Azabicyclo[3.1.0]hex-6-ylmethyl)-N-methyl-2,2-diphenyl-2-propoxyacetamide (Compound No. 43)

¹H NMR (CDCl₃): δ 7.52 (m, 4H), 7.32-7.14 (m, 6H), 3.66-3.27 (m, 4H), 3.00-2.8 (m, 6H), 2.40 (m, 1H), 1.77 (m, 2H), 1.39 (m, 1H), 1.30 (m, 2H), 1.01 (m, 3H).

IR: 3383 and 1643 cm⁻¹; m/z: 379.1 (M⁺+1).

### N-(3-Azabicyclo[3.1.0]hex-6-ylmethyl)-2,2-diphenyl-2-propoxyacetamide (Compound No. 44)

¹H NMR (CDCl₃): δ 7.45 (m, 4H), 7.31 (m, 8H), 3.21-3.14 (m, 2H), 3.03-2.88 (m, 5H), 2.37 (m, 1H), 1.63 (m, 2H), 1.30 (m, 3H), 0.93 (t, J=7.5 Hz, 3H); IR: 3424 and 1674 cm⁻¹; m/z: 365.2 (M⁺+1).

### 3-Azabicyclo[3.1.0]hex-6-ylmethyl hydroxy[bis(4-methylphenyl)]acetate (Compound No. 46)

¹H NMR (CDCl₃): δ 7.31 (d, J=8.0 Hz, 4H), 7.13 (d, J=8.0 Hz, 4H), 4.15 (d, J=7.2 Hz, 2H), 2.85 (m, 4H), 2.34 (s, 6H), 1.37 (m, 2H), 0.96 (m, 1H); m/z: 352.1 (M⁺+1).

### N-(3-Azabicyclo[3.1.0]hex-6-ylmethyl)-2,2-bis(4-fluorophenyl)-2-hydroxy-N-methylacetamide (Compound No. 47)

¹H NMR (CDCl₃): δ 7.35 (m, 4H), 7.07 (m, 4H), 3.46 (m, 1.5H), 3.12-2.56 (m, 8.5H), 1.45 (m, 2H), 0.99 (m, 1H).

### N-(3-Azabicyclo[3.1.0]hex-6-ylmethyl)-2-hydroxy-N-methyl-2,2-bis(4-methylphenyl)acetamide (Compound No. 48)

¹H NMR (CDCl₃): δ 7.25-7.15 (m, 8H), 3.44 (m, 1H), 3.10-2.95 (m, 3H), 2.61-2.54 (m, 3H), 2.35 (s, 6H), 2.00 (m, 2H), 1.50 (m, 1H), 0.9 (m, 2H); m/z: 365 (M⁺+1).

### (2-Methyl-3-azabicyclo[3.1.0]hex-6-yl)methyl cyclohexyl(hydroxy)phenylacetate (Compound No. 64)

¹HNMR (CDCl₃): δ 7.65 (m, 2H), 7.33 (m, 3H), 4.15-3.96 (m, 3H), 3.29 (m, 1H), 3.06-2.92 (m, 2H), 2.4 (m, 1H), 2.21 (m, 2H), 1.8-1.0 (m, 10H).

### N-3-Azabicyclo[3.1.0]hex-6-yl-2-cyclohexyl-N-ethyl-2-hydroxy-2-phenylacetamide (Compound No. 94)

¹HNMR (CDCl₃): δ 7.41-7.16 (m, 5H), 3.37-3.23 (m, 1H), 3.20-3.07 (m, 2H), 2.94-2.91 (m, 2H), 2.43 (m, 1H), 2.24 (m, 1H), 1.80-1.26 (m, 16H).

### N-{3-Azabicyclo[3.1.0]hex-6-ylmethyl}1-N-methyl-2,2-diphenylpropanamide (Compound No. 109)

¹H NMR (CDCl₃): δ 7.37-7.14 (10H, bs), 2.32 (2H, bs), 1.80 (4H, bs), 1.60 (1H, bs), 1.33 (8H, bs), 0.88 (1H, bs); MS: 335 (M⁺+1).

### EXAMPLE 4

### Synthesis of 2-hydroxy-N-[(3-methyl-3-azabicyclo[3.1.0]hex-6-yl)methyl]-2-phenyl-2-(2-thienyl)acetamide (Compound No. 19)

To a solution of the Compound No. 17 (0.2 g, 0.609 mmol) in acetonitrile containing formaldehyde (3 ml), was added sodium cyanoborohydride (0.192 g, 3.0487 mmol) and stirred the reaction mixture at room temperature for 3 hours. It was neutralized with acetic acid and stirred for 30 minutes. The reaction mixture was concentrated under reduced pressure and the residue thus obtained was diluted with water, basified to pH˜14 and extracted with ethyl acetate. The organic layer was washed with water and brine, dried over anhydrous sodium sulphate and concentrated & under reduced pressure to furnish the title compound. The residue thus obtained was purified by column chromatography using 5% methanol in dichloromethane and 1% ammonium solvent mixture to furnish the title compound. Yield: 120 mg.

¹H NMR (CDCl₃): 7.51-7.36 (m, 2H), 7.36-7.26 (m, 4H), 7.06-6.98 (m, 2H), 6.46 (bs, 1H), 3.19-3.14 (m, 2H), 2.96-2.93 (m, 2H), 2.29-2.17 (m, 6H), 1.39 (m, 3H).

Analogs of 2-hydroxy-N-[(3-methyl-3-azabicyclo[3.1.0]hex-6-yl)methyl]-2-phenyl-2-(2-thienyl)acetamide (Compound No. 19) described below, were prepared by using appropriate amine in place of Compound No. 17 and appropriate aldehyde in place of formaldehyde.

### (3-Methyl-3-azabicyclo[3.1.0]hex-6-yl)methyl hydroxy(3-methylphenyl)phenylacetate (Compound No. 25)

¹H NMR (CDCl₃): δ 7.47-7.12 (m, 9H), 4.10 (d, J=6 Hz, 2H), 2.98 (m, 2H), 2.31 (m, 8H), 1.59 (m, 1H), 1.3 (m, 2H); MS: 352.2 (M⁺+1); IR (KBr): 3424 and 1735 cm⁻¹.

### 2-Hydroxy-N-methyl-2,2-diphenyl-N-{[3-(2-thienylmethyl)-3-azabicyclo[3.1.0]hex-6-yl]methyl}acetamide (Compound No. 45)

¹H NMR (CDCl₃): δ 7.38-7.22 (m, 11H), 6.94-6.88 (m, 2H), 3.85-3.67 (m, 2H), 3.43 (m, 1H), 3.14-2.00 (m, 8H), 1.00 (m, 3H); m/z: 433 (M⁺+1).

### 2-Cyclopentyl-2-hydroxy-N-[(3-methyl-3-azabicyclo[3.1.0]hex-6-yl)methyl]-2-phenylacetamide (Compound No. 75)

¹HNMR (CDCl₃): δ 7.60 (m, 2H), 7.31 (m, 3H), 6.40 (bs, 1H), 3.24 (bs, 1H), 3.05 (m, 3H), 2.93 (m, 2H), 2.24 (m, 5H), 1.70-1.55 (m, 9H), 1.24 (2H); IR: 3411 and 1658 cm⁻¹;

MS: 329 (M⁺+1).

### 2-Cyclopentyl-2-hydroxy-2-phenyl-N-{[3-(2-thienylmethyl)-3-azabicyclo[3.1.0]hex-6-yl]methyl}acetamide (Compound No. 81)

¹H NMR (CDCl₃): δ 7.60 (m, 2H), 7.36-7.19 (m, 4H), 6.92 (m, 1H), 6.85 (m, 1H), 6.42 (bs, 1H), 3.77 (s, 2H), 3.18 (bs, 1H), 3.07-2.95 (m, 4H), 2.36 (m, 2H), 1.9-1.75 (m, 7H), 1.30 (m, 4H), 0.89 (m, 1H); IR: 3412 and 1654 cm⁻¹; MS: 411 (M⁺+1).

### EXAMPLE 5

### Synthesis of 2-cyclohexyl-2-hydroxy-N-({3-[(6-methylpyridin-2-yl)methyl]-3-azabicyclo[3.1.0]hex-6-yl}methyl)-2-phenylacetamide (Compound No. 50)

### Step a: 2-(Bromomethyl)-6-methylpyridine

To a solution of the compound (6-methylpyridine-2-yl)methanol (2 g) (commercially available) in carbon tetrachloride (40 ml), was added phosphorous bromide (2.2 g) dropwise and stirred the reaction mixture at 80° C. for 3 hours and then at room temperature for overnight. It was quenched with water and 10% sodium hydroxide solution. The solvent was evaporated under reduced pressure and the aqueous layer was extracted with dichloromethane. The organic layer was collected and washed with 10% sodium hydroxide solution, water and brine, dried over anhydrous sodium sulphate and evaporated under reduced pressure. The residue thus obtained was purified by column chromatography using 10% ethyl acetate in hexane to furnish the title compound.

Yield=1.7 g.

### Step b: 2-Cyclohexyl-2-hydroxy-N-({3-[(6-methylpyridin-2-yl)methyl]-3-azabicyclo[3.1.0]hex-6-yl}methyl)-2-phenyl acetamide

To a solution of the compound N-(3-azabicyclo[3.1.0]hex-6-ylmethyl)-2-cyclohexyl-2-hydroxy-2-phenyl acetamide (disclosed in WO 04/005252) (200 mg) in dry acetonitrile (10 ml), was added 2-(bromomethyl)-6-methylpyridine (136 mg), potassium carbonate (337 mg) and potassium iodide (101 mg) and stirred the mixture 50-60° C. for 3 hours. The reaction mixture was stirred for overnight and quenched by addition of water and ethyl acetate. The organic layer was separated dried over anhydrous sodium sulphate, evaporated under reduced pressure. The residue thus obtained was purified by column chromatography using 2% dichloromethane in methanol to furnish the title compound.

Yield=124 mg.

¹HNMR (CDCl₃): δ 7.60 (m, 2H), 7.53 (m, 1H), 7.36-7.22 (m, 3H), 7.11 (m, 1H), 6.99 (m, 1H), 6.67 (bs, 1H), 3.69 (s, 2H), 3.08-2.94 (m, 4H), 2.52 (s, 3H), 2.40 (m, 3H), 1.7-1.1 (m, 11H), 0.9 (m, 2H); MS: 434 (M⁺+1).

Analogs of 2-cyclohexyl-2-hydroxy-N-({3-[(6-methylpyridin-2-yl)methyl]-3-azabicyclo[3.1.0]hex-6-yl}methyl)-2-phenylacetamide (Compound No. 50) described below were prepared by condensing appropriate amide or ester with an halo compound, respectively, as applicable in each case.

### 2-Cycloheptyl-2-hydroxy-2-phenyl-N-{[3-(pyridin-2-ylmethyl)-3-azabicyclo[3.1.0]hex-6-yl]methyl}acetamide (Compound No. 55)

¹HNMR (CD₃OD): δ 8.66 (m, 1H), 8.08 (m, 1H), 7.8 (m, 1H), 7.64 (m, 1H), 7.30 (m, 5H), 4.98 (m, 2H), 4.17 (m, 1H), 3.80 (m, 1H), 3.10 (m, 2H), 2.66 (m, 1H), 2.33 (m, 1H), 1.96-0.8 (m, 16H); IR (KBr): 3406 and 1653 cm⁻¹; MS: 434 (M⁺+1).

### 2-Cyclopentyl-2-hydroxy-N-({3-[(6-methylpyridin-2-yl)methyl]-3-azabicyclo[3.1.0]hex-6-yl}methyl)-2-phenylacetamide (Compound No. 56)

¹HNMR (CDCl₃): δ 7.62 (m, 2H), 7.54 (m, 1H), 7.34 (m, 2H), 7.27 (m, 1H), 7.12 (m, 1H), 7.00 (m, 1H), 6.44 (bs, 1H), 3.69 (s, 2H), 3.20 (bs, 1H), 3.10-2.95 (m, 5H), 2.52 (s, 3H), 2.40 (m, 2H), 1.70-1.2 (m, 11H); MS: 420 (M⁺+1).

### 2-Hydroxy-N-{[3-(3-methylbut-2-en-1-yl)-3-azabicyclo[3.1.0]hex-6-yl]methyl}-2,2-diphenylacetamide (Compound No. 71)

¹HNMR (CDCl₃): δ 7.25 (m, 10H), 6.79 (bs, 1H), 5.32 (bt, 1H), 3.96 (m, 1H), 3.51 (m, 4H), 3.12 (m, 2H), 3.0 (m, 1H), 1.69-1.44 (m, 7H), 0.76 (m, 2H); MS: 391 (M⁺+1); IR: 3408 and 1660 cm⁻¹.

### 2-Hydroxy-N-{[3-(4-methylpent-3-en-1-yl)-3-azabicyclo[3.1.0]hex-6-yl]methyl}-2,2-diphenylacetamide (Compound No. 72)

¹HNMR (CDCl₃): δ 7.36 (m, 10H), 6.93 (bs, 1H), 4.98 (m, 1H), 3.82 (m, 2H), 3.24 (m, 2H), 2.90 (m, 3H), 2.55 (m, 2H), 1.70 (m, 10H); IR: 3406 and 1659 cm⁻¹; MS: 405 (M⁺+1).

### 2-Cycloheptyl-2-hydroxy-2-phenyl-N-{[3-(pyridin-3-ylmethyl)-3-azabicyclo[3.1.0]hex-6-yl]methyl}acetamide (Compound No. 73)

¹HNMR (CD₃OD): δ 8.44 (m, 2H), 7.76 (m, 2H), 7.6 (m, 2H), 7.20 (m, 3H), 5.47 (bs, 1H), 3.70 (m, 2H), 2.90 (m, 4H), 2.53 (m, 3H), 1.53-1.28 (m, 15H); IR: 3441 and 1644 cm⁻¹; MS: 434 (M⁺+1).

### 2-Cycloheptyl-2-hydroxy-2-phenyl-N-{[3-(pyridin-4-ylmethyl)-3-azabicyclo[3.1.0]hex-6-yl]methyl}acetamide (Compound No. 74)

¹HNMR (CDCl₃): δ 8.52 (m, 2H), 7.60 (m, 2H), 7.30 (m, 5H), 6.69 (bs, 1H), 3.57 (m, 2H), 3.0-2.9 (m, 5H), 2.6 (m, 1H), 2.34 (m, 3H), 1.85-1.6 (m, 11H); IR: 3417 and 1650 cm⁻¹; MS: 434 (M⁺+1).

### 2-Cyclopentyl-2-hydroxy-2-phenyl-N-{[3-(2-phenylethyl)-3-azabicyclo[3.1.0]hex-6-yl]methyl}acetamide (Compound No. 76)

¹HNMR (CDCl₃): δ 7.60 (m, 2H), 7.37-7.15 (m, 8H), 6.42 (bs, 1H), 3.21 (bs, 1H), 3.05 (m, 5H), 2.65 (m, 4H), 2.30 (m, 2H), 1.72-1.56 (m, 9H), 1.25 (m, 2H).

### N-{[3-(1,3-Benzodioxol-5-ylmethyl)-3-azabicyclo[3.1.0]hex-6-yl]methyl}-2-cyclohexyl-2-hydroxy-2-phenylacetamide (Compound No. 80)

¹HNMR (CD₃OD): δ 7.58 (m, 2H), 7.25 (m, 3H), 6.88 (m, 3H), 5.99 (s, 2H), 4.41 (s, 2H), 4.09 (m, 2H), 3.3-3.2 (m, 3H), 3.04 (m, 2H), 2.5 (m, 1H), 1.68 (m, 6H), 1.25 (m, 7H).

### 2-Cyclohexyl-2-hydroxy-2-phenyl-N-{[3-(2-phenylethyl)-3-azabicyclo[3.1.0]hex-6-yl]methyl}acetamide (Compound No. 82)

¹HNMR (CDCl₃): δ 7.60 (m, 2H), 7.29 (m, 8H), 6.88 (bs, 1H), 3.41 (m, 2H), 3.14-2.92 (m, 7H), 2.69 (m, 2H), 2.41 (m, 1H), 1.78-0.88 (m, 13H); MS: 433 (M⁺+1); IR: 3409 and 1652 cm⁻¹.

### N-({3-[2-(1,3-Benzodioxol-5-yl)ethyl]-3-azabicyclo[3.1.0]hex-6-yl}methyl)-2-cycloheptyl-2-hydroxy-2-phenylacetamide (Compound No. 83)

¹HNMR (CDCl₃): δ 7.60 (m, 2H), 7.30 (m, 4H), 6.95 (bs, 1H), 6.69 (m, 2H), 5.93 (s, 2H), 3.50 (m, 2H), 3.15-2.64 (m, 9H), 1.71-1.11 (m, 14H), 0.88 (m, 2H); IR (KBr): 3407 and 1650 cm⁻¹; MS: 491 (M⁺+1).

### 2-Cycloheptyl-N-({3-[2-(2,3-dihydro-1-benzofuran-5-yl)ethyl]-3-azabicyclo[3.1.0]hex-6-yl}methyl)-2-hydroxy-2-phenylacetamide (Compound No. 84)

¹HNMR (CDCl₃): δ 7.60 (m, 2H), 7.33 (m, 3H), 7.05 (m, 1H), 7.01 (bs, 1H), 6.91 (m, 1H), 6.69 (m, 1H), 4.54 (t, 2H, J=8.7 Hz), 3.48 (m, 2H), 3.2-2.9 (m, 8H), 2.74-2.63 (m, 3H), 1.75-1.0 (m, 15H); MS: 489 (M⁺+1); IR: 3413 and 1654 cm⁻¹.

### 2-Cyclopentyl-N-{[3-(cyclopropylmethyl)-3-azabicyclo[3.1.0]hex-6-yl]methyl}-2-hydroxy-2-phenylacetamide (Compound No. 92)

¹HNMR (CDCl₃): δ 7.60 (m, 2H), 7.32 (m, 3H), 6.42 (bs, 1H), 3.23 (bs, 1H), 3.05 (m, 4H), 2.27 (m, 4H), 1.88 (m, 1H), 1.71-1.50 (m, 6H), 1.3-1.23 (m, 5H), 0.8 (m, 1H), 0.45 (m, 2H), 0.06 (m, 2H); IR: 3414 and 1660 cm⁻¹; MS: 368 (M⁺+1).

### [3-(1,3-Benzodioxol-5-ylmethyl)-3-azabicyclo[3.1.0]hex-6-yl]methyl cyclohexyl(hydroxy)phenylacetate (Compound No. 99)

¹HNMR (CDCl₃): δ 7.65 (m, 2H), 7.30 (m, 3H), 6.70 (m, 3H), 5.94 (s, 2H), 4.10 (m, 1H), 3.93 (m, 1H), 3.71 (bs, 1H), 3.47 (s, 2H), 2.90 (m, 2H), 2.29 (m, 3H), 1.75 (m, 1H), 1.64-1.12 (m, 12H); IR (cm⁻¹); 3507, 1720 cm⁻¹; m/z: 463 (M⁺+1).

### 2-Hydroxy-2,2-diphenyl-N-{[3-(2-phenylethyl)-3-azabicyclo[3.1.0]hex-6-yl]methyl}acetamide (Compound No. 100)

¹HNMR (CDCl₃): δ 7.46-7.16 (m, 15H), 3.82-3.78 (m, 2H), 3.28-3.22 (m, 6H), 3.01-2.95 (m, 2H), 1.26-1.11 (m, 3H).

### N-({3-[2-(1,3-benzodioxol-5-yl)ethyl]-3-azabicyclo[3.1.0]hex-6-yl}methyl)-2-cyclopentyl-2-hydroxy-N-methyl-2-phenylacetamide (Compound No. 102)

¹HNMR (CDCl₃): δ 7.35-7.24 (m, 5H), 6.72-6.66 (m, 3H), 5.91 (s, 2H), 3.08-2.38 (m, 15H), 1.94-1.25 (m, 11H).

### 2-Hydroxy-N-methyl-N-{[3-(4-methylpent-3-en-1-yl)-3-azabicyclo[3.1.0]hex-6-yl]methyl}-2,2-diphenylacetamide (Compound No. 106)

¹HNMR (CDCl₃): δ 7.43-7.31 (10H, bm), 5.86 (1H, bs), 5.30 (1H, bs), 3.76-3.65 (2H, m), 3.46 (2H, d, 8 Hz), 3.10 (1H, bs), 2.88 (3H, bs), 2.63 (3H, s), 2.53-2.43 (4H, m), 2.04 (1H, s), 1.70 (3H, s), 1.66 (3H, s); MS: 419 (M⁺+1).

### N-({3-[2-(1,3-Benzodioxol-5-yl)ethyl]-3-azabicyclo[3.1.0]hex-6-yl}methyl)-2-hydroxy-N-methyl-2,2-diphenylacetamide (Compound No. 107)

¹HNMR (CDCl₃): δ 7.39-7.33 (10H, bs), 6.73-6.62 (3H, m), 5.92 (2H, s), 3.15-2.18 (13H, m), 1.11 (2H, s); MS: 485 (M⁺+1).

### N-({3-[2-(2,3-Dihydro-1-benzofuran-5-yl)ethyl]-3-azabicyclo[3.1.0]hex-6-yl}methyl)-2-hydroxy-N-methyl-2,2-diphenylacetamide (Compound No. 108)

¹HNMR (CDCl₃): δ 7.43-7.33 (bs, 10H), 7.05 (1H, s), 6.92 (1H, d, 8 Hz), 6.70 (1H, d, 8 Hz), 5.97 (1H, bs), 4.54 (2H, t, 8 Hz), 3.46-2.44 (15H, m), 2.05 (1H, s), 1.26 (2H, m).

MS: 483 (M⁺+1).

### Scheme II

### EXAMPLE 6

### Synthesis of ((3-benzyl-3-azabicyclo[3.1.0]hex-6-yl)methyl methoxy(diphenyl)acetate (Compound No. 31)

The title compound was synthesized following the procedure as described in Example 2, by using 3-benzyl-6-(bromomethyl)-3-azabicyclo[3.1.0]hexane in place of 3-benzyl-3-azabicyclo[3.1.0]hex-6-ylmethyl methane sulfonate and using methoxy (diphenyl acetic acid) in place of hydroxy (phenyl) 2-thienylacetic acid. Yield: 290 mg.

¹H NMR (CDCl₃): δ 7.46 (m, 4H), 7.28 (m, 11H), 4.03 (d, J=7.5 Hz, 2H), 3.54 (s, 2H), 3.17 (s, 3H), 2.84 (m, 2H), 2.27 (m, 2H), 1.30 (m, 2H), 0.88 (m, 1H).

The analogs of (3-benzyl-3-azabicyclo[3.1.0]hex-6-yl)methyl methoxy(diphenyl)acetate (Compound No. 31) described below, were prepared by coupling appropriate acid with halogenated compound, respectively, as applicable in each case.

### (3-benzyl-3-azabicyclo[3.1.0]hex-6-yl)methyl hydroxy(phenyl)-3-thienylacetate (Compound No. 4)

¹H NMR (CDCl₃): δ 7.48-7.14 (m, 13H), 4.31 (s, 1H), 4.15-4.07 (m, 2H), 3.59 (s, 2H), 2.95-2.92 (d, 2H), 2.35-2.33 (d, 2H), 1.34 (bs, 3H); IR (DCM): 1727 cm⁻¹.

### EXAMPLE 7

### Synthesis of 3-azabicyclo[3.1.0]hex-6-ylmethyl methoxy(diphenyl)acetate (Compound No. 35)

The title compound was prepared by following the procedure as described in Example 3, by deprotecting Compound No. 31 in place of Compound No. 29. Yield: 126 mg.

¹H NMR (CDCl₃): δ 7.48-7.33 (m, 10H), 4.11 (d, J=7.2 Hz, 2H), 3.18 (s, 3H), 2.83 (m, 4H), 1.30 (m, 2H), 0.9 (m, 1H); IR: 3423 and 1736 cm⁻¹; MS: 338 cm⁻¹.

### EXAMPLE 8

### Synthesis of 3-azabicyclo[3.1.0]hex-6-ylmethyl (4E or 4Z)-2-hydroxy-2-phenylhex-4-enoate (Compound No. 114)

To a solution of the Compound No. 120 (022 g) in methanol (5 ml) was added etheral hydrochloric acid (10 ml) and stirred the mixture at room temperature for 3 hours. The mixture was concentrated under reduced pressure and the residue thus obtained was diluted with water. The aqueous layer was washed with diethyl ether to remove the impurities. The aqueous layer was basified with aqueous sodium hydroxide. The aqueous layer was extracted with ethyl acetate. The organic layer was separated and washed with water and brine, dried over anhydrous sodium sulphate and concentrated under reduced pressure to furnish the title compound. Yield: 60 mg.

¹HNMR (CDCl₃): δ 7.62 (2H, t, 8 Hz), 7.37-7.29 (3H, m), 5.62 (1H, m), 5.42 (1H, m), 4.07 (2H, d, 4 Hz), 2.97-2.86 (5H, m), 2.68 (1H, m), 1.67 (3H, d, 4 Hz), 1.39-1.00 (3H, m); MS: 302 (M⁺+1).

### Scheme III

### EXAMPLE 9

### Synthesis of tert-butyl (6-{[6-({[cyclohexyl(hydroxy)phenylacetyl]amino}methyl)-3-azabicyclo[3.1.0]hex-3-yl]methyl}pyridin-2-yl)carbamate (Compound No. 65)

### Step a: 6-(Methoxycarbonyl)pyridine-2-carboxylic acid

To a suspension of pyridine-2,6-dicarboxylic acid (20 g) in aqueous methanol (1:1, 200 ml) at 0° C., was added concentrated sulphuric acid (10 ml) dropwise under constant stirring. The mixture was warmed to room temperature and refluxed for 15 minutes. The contents of the reaction mixture were poured into saturated sodium bicarbonate solution and extracted with dichloromethane. The aqueous layer was acidified with concentrated hydrochloric acid (pH=2) and extracted with dichloromethane. The organic layer was combined and evaporated under reduced pressure to furnish the title compound. Yield=7.2 g.

### Step b: Methyl 6-[(tert-butoxycarbonyl)amino]pyridine-2-yl]carboxylate

To a solution of the compound obtained from step a above (5.9 g) in toluene (100 ml), was added triethyl amine (9 ml), hydroxybenzotriazole (20 ml) and diphenylphosphoric azide (12.5 g). The mixture was treated at 100° C. for overnight and subsequently cooled to room temperature. The reaction mixture was diluted with ethyl acetate and aqueous sodium bicarbonate solution. The organic layer was separated, washed with water and brine, dried over anhydrous sodium sulphate and evaporated under reduced pressure. The residue thus obtained was purified by column chromatography using 10% ethylacetate in hexane solvent mixture as eluent to furnish the title compound. Yield=3.8 g.

### Step c: Tert-butyl [6-(hydroxymethyl)pyridin-2-yl]carbamate]

The compound obtained from step b above (3.8 g) was dissolved in ethanol (75 ml) and calcium chloride (3.34 g) was added. The mixture was cooled to 0° C. followed by the addition of sodium cyanoborohydride (2.98 g) slowly. The reaction mixture was stirred for 2 hours at 0° C. The solvent was evaporated under reduced pressure and the residue thus obtained was partitioned between water and ethyl acetate. The organic layer was dried over anhydrous sodium sulphate, evaporated under reduced pressure to furnish the title compound. Yield=3.4 g.

### Step d: {6-[(tert-butoxycarbonyl)amino]pyridin-2-yl}methyl methanesulfonate

To a solution of the compound obtained from step c above (3.4 g) in ethyl acetate (80 ml) at 0° C., was added triethyl amine (7.4 ml) and methane sulphonyl chloride (3.5 ml) and stirred the mixture for 2 hours. The reaction mixture was diluted with ethyl acetate, washed with saturated sodium bicarbonate solution, water and brine and dried over anhydrous sodium sulphate. The solvent was evaporated under reduced pressure to furnish the title compound. Yield=3.4 g.

### Step e: Tert-butyl (6-{[6-Cyclohexyl(hydroxy)phenylacetyl]amino}methyl)-3-azabicyclo[3.1.0]hex-3-yl]methyl}pyridin-2-yl)carbamate

To a solution of the compound N-(3-azabicyclo[3.1.0]hex-6-ylmethyl)-2-cyclohexyl-2-hydroxy-2-penylacetamide (disclosed in WO 04/005252) (1.19 g) in acetonitrile (30 ml), was added the compound obtained from step d above (1.32 g) and potassium carbonate (2 g). The reaction mixture was stirred for overnight at room temperature. The solvent was evaporated under reduced pressure and the residue thus obtained was partitioned between ethylacetate and water. The organic layer was collected, washed with water and brine, dried over anhydrous sodium sulphate and evaporated under reduced pressure. The residue thus obtained was purified by column chromatography to furnish the title compound. Yield=1.08 g.

¹H NMR (CDCl₃): δ 7.76 (m, 1H), 7.59 (m, 3H), 7.36-7.22 (m, 3H), 6.92 (m, 1H), 6.66 (bs, 1H), 3.56 (s, 2H), 3.07 (m, 1H), 2.93 (m, 4H), 2.36 (m, 3H), 1.70 (m, 5H), 1.51 (s, 9H), 1.37-1.16 (m, 5H), 0.91 (m, 3H); Mass (m/z): 535 (M⁺+1).

Analogs of tert-butyl (6-{[6-({[cyclohexyl(hydroxy)phenylacetyl]amino}methyl)-3-azabicyclo[3.1.0]hex-3-yl]methyl}pyridin-2-yl)carbamate (Compound No. 65) described below were prepared by using appropriate acid in place of cyclohexyl(hydroxy)phenylacetic acid, respectively, as applicable in each case.

### Tert-butyl (6-{[6-({[hydroxy(diphenyl)acetyl]amino}methyl)-3-azabicyclo[3.1.0]hex-3-yl]methyl}pyridin-2-yl)carbamate (compound no. 66)

¹HNMR (CDCl₃): δ 7.78-7.57 (d, 1H, J=8.4 Hz), 7.62-7.59 (t, 1H, J=7.8 Hz), 7.44-7.31 (m, 10H), 6.94-6.92 (d, 1H, J=7.2 Hz), 6.36 (bs, 1H), 3.5 (s, 2H), 3.18-3.14 (t, 2H), 2.98 (m, 2H), 2.40 (m, 2H), 2.04 (s, 1H), 1.47 (s, 9H), 1.29-1.24 (m, 2H).

### Tert-butyl (6-{[6-({[cyclopentyl(hydroxy)phenylacetyl]amino}methyl)-3-azabicyclo[3.1.0]hex-3-yl]methyl}pyridin-2-yl)carbamate (Compound No. 67)

¹HNMR (CDCl₃): δ7.78-7.75 (d, 1H, 9 Hz), 7.61-7.56 (m, 3H), 7.36-7.25 (m, 3H), 6.94-6.91 (d, 1H, J=9 Hz), 3.456 (s, 2H), 3.22 (bs, 1H), 3.07-2.92 (m, 5H), 2.37-2.35 (m, 2H), 1.72-1.24 (m, 19H).

### EXAMPLE 10

### Synthesis of N-({3-[(6-aminopyridin-2-yl)methyl]-3-azabicyclo[3.1.0]hex-6-yl}methyl)-2-cyclohexyl-2-hydroxy-2-phenylacetamide (Compound No. 52)

A solution of the Compound No. 65 (140 mg) and methanolic hydrochloric acid (10 ml) was stirred for overnight at room temperature. The solvent was evaporated under reduced pressure and the residue thus obtained was partitioned between aqueous sodium bicarbonate and ethyl acetate. The organic layer was separated, dried over anhydrous sodium sulphate and evaporated under reduced pressure to furnish the title compound. Yield=105 mg.

¹HNMR (CDCl₃): δ 7.61 (m, 2H), 7.32 (m, 5H), 7.0 (m, 1H), 4.20 (m, 2H), 3.5-3.35 (m, 3H), 3.12 (m, 2H), 2.39 (m, 1H), 1.96 (m, 1H), 1.76-1.64 (m, 5H), 1.2 (m, 6H), 0.9 (m, 2H); MS: 435 (M⁺+1).

Analogs of N-({3-[(6-aminopyridin-2-yl)methyl]-3-azabicyclo[3.1.0]hex-6-yl}methyl)-2-cyclohexyl-2-hydroxy-2-phenylacetamide (Compound No. 52) described below were prepared similarly.

### N-({3-[(6-aminopyridin-2-yl)methyl]-3-azabicyclo[3.1.0]hex-6-yl}methyl)-2-cyclopentyl-2-hydroxy-2-phenylacetamide (Compound No. 51)

¹HNMR (CDCl₃): δ 7.60 (m, 2H), 7.30 (m, 4H), 6.63 (m, 1H), 6.45 (bs, 1H), 6.35 (m, 1H), 4.73 (bs, 2H), 3.53 (s, 2H), 3.03 (m, 5H), 2.37 (m, 2H), 1.71-1.23 (m, 11H); MS: 421 (M⁺+1).

### N-({3-[(6-aminopyridin-2-yl)methyl]-3-azabicyclo[3.1.0]hex-6-yl}methyl)-2-hydroxy-2,2-diphenylacetamide (Compound no. 57)

¹HNMR (CDCl₃): δ 7.40 (m, 11H), 6.64 (m, 1H), 6.36 (m, 2H), 4.39 (bs, 2H), 3.53 (s, 2H), 3.15 (m, 2H), 3.01 (m, 2H), 2.38 (m, 2H), 1.29 (m, 3H); MS: 429 (M⁺+1).

### EXAMPLE 11

### Synthesis of tartarate salt of N-(3-azabicyclo[3.1.0]hex-6-ylmethyl)-3-hydroxy-N-methyl-3,3-diphenylpropanamide (Compound No. 37)

### Step a: N-(3-Azabicyclo[3.1.0]hex-6-ylmethyl)-3-hydroxy-N-methyl-3,3-diphenyl propionamide

The title compound was prepared following the procedure as described in Example 3, by using compound (Compound No. 23) in place of (Compound No. 29).

Yield: 249 mg.

### Step b: Tartarate salt of N-(3-azabicyclo[3.1.0]hex-6-ylmethyl)-3-hydroxy-N-methyl-3,3-diphenylpropanamide

A solution of the compound obtained from step a above (249 mg) and tartaric acid (106 g) in ethanol (5 ml) were heated at 60-70° C. for 40 minutes. The reaction mixture was concentrated under reduced pressure and the residue thus obtained was triturated with ether. The etheral layer was decanted off and the residue thus obtained was dried under reduced pressure. Yield: 90 mg.

¹H NMR (CDCl₃): 7.39 (m, 4H), 7.28-7.15 (m, 6H), 4.35 (s, 2H), 3.37-3.21 (m, 7H), 3.04 (m, 3H), 2.84 (m, 1H), 1.55 (m, 2H), 0.95 (m, 1H).

IR (KBr): 3447.2 and 1615.3 cm⁻¹.

The analogs of N-(3-azabicyclo[3.1.0]hex-6-ylmethyl)-3-hydroxy-N-methyl-3,3-diphenylpropanamide (Compound No. 37) described below, were prepared similarly.

### N-[(3-Benzyl-3-azabicyclo[3.1.0]hex-6-yl)methyl]-2-cyclopentyl-2-hydroxy-2-(2-thienyl)acetamide (Compound No. 7)

¹H NMR (CDCl₃): δ 7.31-7.21 (m, 6H), 7.08-7.07 (m, 1H), 6.97-6.94 (m, 1H), 6.35 (bs, 1H), 3.69 (s, 1H), 3.57 (s, 2H), 3.10-3.06 (m, 2H), 2.94-2.82 (m, 2H), 2.79 (m, 1H), 2.33-2.31 (m, 2H), 1.63-1.25 (m, 11H); IR (KBr): 1657 cm⁻¹

### Tartarate salt of N-(3-benzyl-3-azabicyclo[3.1.0]hex-6-yl)-2-cyclopentyl-2-hydroxy-2-(2-thienyl)acetamide (Compound No. 9)

¹H NMR (CDCl₃): δ 7.29-7.17 (m, 6H), 7.06-7.04 (m, 1H), 6.97-6.94 (m, 1H), 6.3 (bs, 1H), 3.64 (bs, 1H), 3.54 (s, 2H), 3.09-3.02 (m, 3H), 2.80-2.75 (m, 1H), 2.37-2.35 (d, 2H), 1.63-1.28 (m, 10H); IR (DCM): 1655 cm⁻¹.

### Tartarate salt of N-(3-azabicyclo[3.1.0]hex-6-ylmethyl)-2-cyclopentyl-2-hydroxy-2-(2-thienyl)acetamide (Compound No. 17)

¹H NMR (CDCl₃): δ 7.33-7.22 (m, 6H), 7.08-7.07 (m, 1H), 6.97-6.94 (m, 1H), 6.51 (bs, 1H), 5.09 (s, 2H), 3.62-3.4 (m, 3H), 3.22-3.12 (m, 4H), 2.87-2.82 (m, 1H), 1.64-1.25 (m, 10H), 0.79 (m, 1H).

### Tartarate salt of N-({3-[(6-aminopyridin-2-yl)methyl]-3-azabicyclo[3.1.0]hex-6-yl}methyl)-2-cyclopentyl-2-hydroxy-2-phenylacetamide (compound No. 53)

¹HNMR (DMSO-d₆): δ 7.58 (m, 2H), 7.30 (m, 4H), 6.44 (m, 1H), 6.33 (m, 1H), 5.91 (bs, 2H), 4.19 (s, 2H), 3.5 (m, 5H dried under water in DMSO peaks), 3.0 (m, 4H), 1.52-1.23 (m, 11H); HPLC: 91.41% (24.7 min).

### Tartarate salt of N-({3-[(6-aminopyridin-2-yl)methyl]-3-azabicyclo[3.1.0]hex-6-yl}methyl)-2-cyclohexyl-2-hydroxy-2-phenylacetamide (Compound No. 54)

¹HNMR (CD₃OD): δ 7.64-7.53 (m, 3H), 7.35-7.21 (m, 3H), 6.82 (m, 1H), 6.67 (m, 1H), 4.57 (s, 2H), 4.04 (s, 2H), 3.36 (m, 4H), 3.18-3.0 (m, 5H), 2.4 (m, 1H), 1.76-1.6 (m, 5H), 1.20 (m, 8H); HPLC: 95.12% (12.6 min.).

### Tartarate salt of N-[(3-benzyl-2-methyl-3-azabicyclo[3.1.0]hex-6-yl)methyl]-2-cyclohexyl-2-hydroxy-2-phenylacetamide (Compound No. 63)

¹HNMR (CD₃OD): δ 7.53 (m, 2H), 7.35-7.13 (m, 8H), 4.35 (s, 2H), 4.24 (m, 2H), 3.89 (m, 1H), 3.51-3.41 (m, 4H), 3.10 (m, 2H), 2.34 (m, 1H), 1.62-1.40 (m, 6H), 1.28-0.80 (m, 8H); HPLC: 97.07% (16.5 min).

### Tartarate salt of [3-(4-methylpent-3-en-1-yl)-3-azabicyclo[3.1.0]hex-6-yl]methyl cyclopentyl(hydroxy)phenylacetate (Compound No. 68)

¹HNMR (DMSO-d₆): δ 7.65 (m, 2H), 7.34 (m, 3H), 5.7 (bs, 1H), 5.11 (bt, 1H), 4.25 (s, 2H), 4.07-3.0 (peaks superceded by DMSO-H₂O peaks), 2.6 (m, 2H), 2.19 (m, 1H), 1.72-1.26 (m, 10H); IR (KBr): 3322 and 1722 cm⁻¹.

### Tartarate salt of [3-(1-phenylethyl)-3-azabicyclo[3.1.0]hex-6-yl]methyl cyclohexyl(hydroxy)phenylacetate (Compound No. 69)

¹HNMR (DMSO-d₆): δ 7.56 (m, 2H), 7.28 (m, 8H), 5.50 (bs, 1H), 4.25 (s, 2H), 4.0-3.0 (peaks superscripted by H₂O in DMSO peaks), 2.13 (m, 3H), 1.55-1.03 (m, 14H); IR (KBr): 3485 and 1723 cm⁻¹.

### Tartarate salt of N-[(3-benzyl-3-azabicyclo[3.1.0]hex-6-yl)methyl]-2-cyclobutyl-2-hydroxy-2-phenylacetamide (Compound No. 70)

¹HNMR (CD₃OD): δ 7.40 (m, 2H), 7.30 (m, 5H), 7.12 (m, 3H), 4.32 (s, 2H), 4.04 (s, 2H), 3.35 (m, 1H), 3.20 (m, 4H), 2.98 (m, 2H), 2.04-1.6 (m, 7H), 1.20 (m, 2H); HPLC: 98.73 (12.9 min).

### Tartarate salt of [3-(3-methylbut-2-en-1-yl)-3-azabicyclo[3.1.0]hex-6-yl]methyl cyclopentyl(hydroxy)phenylacetate (Compound No. 87)

¹HNMR (CD₃OD): δ 7.62 (m, 2H), 7.30 (m, 3H), 5.25 (m, 1H), 4.40 (s, 2H), 4.03 (m, 2H), 3.70 (m, 2H), 3.35 (m, 4H), 2.98 (m, 1H), 1.81-1.74 (m, 8H), 1.60 (m, 5H), 1.40 (m, 4H); IR: 3319 and 1724 cm⁻¹.

### Tartarate salt of [3-(1,3-benzodioxol-5-ylmethyl)-3-azabicyclo[3.1.0]hex-6-yl]methyl cyclopentyl(hydroxy)phenylacetate (Compound No. 88)

¹HNMR (CD₃OD): δ 7.60 (m, 2H), 7.29 (m, 2H), 7.21 (m, 1H), 6.88 (m, 3H), 5.99 (s, 2H), 4.42 (s, 2H), 4.05-3.97 (m, 3H), 3.33-3.17 (m, 5H), 3.0 (m, 1H), 1.72-1.26 (m, 11H); IR (KBr): 3484 and 1723 cm⁻¹.

### Tartarate salt of N-[(3-benzyl-3-azabicyclo[3.1.0]hex-6-yl)methyl]-2-cyclohexyl-2-hydroxy-N-methyl-2-phenylacetamide (Compound No. 89)

¹HNMR (CD₃OD): δ 7.43-7.21 (m, 10H), 4.43 (s, 2H), 4.21 (m, 2H), 3.4-3.1 (m, 7H), 2.88 (m, 2H), 2.26 (m, 1H), 1.86-1.73 (m, 3H), 1.61 (m, 2H), 1.36-0.95 (m, 8H); IR (KBr): 3424, 1735 and 1617 cm⁻¹; MS: 433 (M⁺+1).

### Tartarate salt of N-[(3-benzyl-3-azabicyclo[3.1.0]hex-6-yl)methyl]-2-hydroxy-N-methyl-2,2-diphenylacetamide (Compound No. 90)

¹HNMR (CD₃OD): δ 7.35 (m, 5H), 7.19 (m, 10H), 4.32 (s, 2H), 4.17-4.10 (m, 2H), 3.30 (m, 3H), 3.20 (m, 4H), 2.87 (m, 1H), 2.76 (m, 2H), 1.74 (m, 1H), 1.3 (m, 1H), 1.2 (m, 1H); IR: 3424, 1735 and 1625 cm⁻¹.

### Tartarate salt of (3-benzyl-3-azabicyclo[3.1.0]hex-6-yl)methyl cyclopentyl(hydroxy)-2-thienylacetate (Compound No. 95)

¹HNMR (CD₃OD): δ 7.37 (m, 5H), 7.21 (m, 1H), 7.07 (m, 1H), 6.89 (m, 1H), 4.38 (s, 2H), 4.02 (m, 4H), 3.15 (m, 4H), 2.81 (m, 1H), 1.68-1.4 (m, 11H).

### Tartarate salt of N-[(3-benzyl-3-azabicyclo[3.1.0]hex-6-yl)methyl]-2-cyclopentyl-2-hydroxy-N-methyl-2-phenylacetamide (Compound No. 103)

¹H NMR (CD₃OD): δ 7.46-7.21 (m, 10H), 4.49 (s, 2H), 4.29 (m, 2H), 3.5-3.0 (m, 6H), 2.83 (m, 4H), 1.83 (m, 2H), 1.52-1.28 (m, 9H); IR (KBr): 3422, 1736 and 1619 cm⁻¹.

### EXAMPLE 12

### Radioligand Binding Assays

The affinity of test compounds for M₁, M₂ and M₃ muscarinic receptor subtypes was determined by [³H]-N-methylscopolamine binding studies using rat heart and submandibular gland respectively as described by Moriya et al., (Life Sci., 1999, 64(25): 2351-2358) with minor modifications. In competition binding studies, specific binding of [3H] NMS was also determined using membranes from Chinese hamster ovary (CHO) cells expressing cloned human M₁, M₂, M₃, M₄ and M₅ receptors. Selectivities were calculated from the Ki values obtained on these human cloned membranes.

Membrane preparation: Submandibular glands and heart were isolated and placed in ice-cold homogenizing buffer (HEPES 20 mM, 10 mM EDTA, pH 7.4) immediately after sacrifice. The tissues were homogenized in 10 volumes of homogenizing buffer and the homogenate was filtered through two layers of wet gauze and filtrate was centrifuged at 500 g for 10 min. The supernatant was subsequently centrifuged at 40,000 g for 20 min. The pellets thus obtained were resuspended in assay buffer (HEPES 20 mM, EDTA 5 mM, pH 7.4) and were stored at −70° C. until the time of assay.

Ligand binding assay: The compounds were dissolved and diluted in DMSO. The membrane homogenates (150-250 μg protein) were incubated in 250 μl of assay volume (HEPES 20 mM, pH 7.4) at 24-25° C. for 3 h. Non-specific binding was determined in the presence of 1 μM atropine. The incubation was terminated by vacuum filtration over GF/B fiber filters (Wallac). The filters were then washed with ice-cold 50 mM Tris HCT buffer (pH 7.4). The filter mats were dried and bound radioactivity retained on filters was counted. The IC₅₀ & K_{d} were estimated by using the non-linear curve fitting program using G Pad Prism software. The value of inhibition constant Kᵢ was calculated from competitive binding studies by using Cheng & Prusoff equation (Biochem Pharmacol, 1973, 22: 3099-3108), Ki=IC₅₀/(1+L/Kd), where L was the concentration of [³H]NMS used in the particular experiment. pki is −log [Ki].

### Functional Experiments Using Isolated Rat Bladder:

### Methodology:

Animals were euthanized by overdose of thiopentone and whole bladder was isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder was cut into longitudinal strips (3 mm wide and 5-6 mm long) and mounted in 10 ml organ baths at 30° C., with one end connected to the base of the tissue holder and the other end connected through a force displacement transducer. Each tissue was maintained at a constant basal tension of 1 g and allowed to equilibrate for 1^{1/2} hour during which the Tyrode buffer was changed every 15-20 min. At the end of equilibration period the stabilization of the tissue contractile response was assessed with 1 μmol/L of Carbachol until a reproducible response was obtained. Subsequently a cumulative concentration response curve to carbachol (10⁻⁹ mol/L to 3×10⁻⁴ mol/L) was obtained. After several washes, once the baseline was achieved, cumulative concentration response curve was obtained in presence of NCE (NCE added 20 min. prior to the second cumulative response curve.

The contractile results were expressed as % of control E max. ED50 values were calculated by fitting a non-linear regression curve (Graph Pad Prism). pKb values are were where,

dose ratio=ED50 in the presence of antagonist/ED50 in the absence of antagonist.

Compounds specifically described herein displayed Ki for rat M3 receptors of between about 1000 and about 0.1 nM, for example, between about 200 and about 0.1 nM, or for example between about 50 and about 0.1 nM, or for example between about 15 and about 0.1 nM, or for example between about 8 and about 0.1 nM, or for example between about 1 and about 0.1 nM. Compounds specifically described herein displayed Ki for rat M2 receptors of between about 1000 and about 0.15 nM, for example, between about 200 and about 0.15 nM, or for example between about 50 and about 0.15 nM, or for example between about 15 and about 0.15 nM, or for example between about 8 and about 0.15 nM, or for example between about 1 and about 0.15 nM. Based on such measurements, the compounds described herein displayed selectivity (Ki for rat M3 receptors/Ki for rat M2 receptors) of between about 0.3 and about 310, for example between about 10 and about 310, or for example between about 30 and about 310, or for example between about 60 and about 310.

Particular compounds (Nos. 51, 53, 54, 58-60, 63, 68-70, 80, 87-90, 99 and 103) were tested for pKb, and gave values from about 7.4 to about 8.6.

Particular compounds (Nos. 2-4, 6-8, 10-12, 14-19, 25, 27, 35, 95, 104-106, 108, 110-112, 114-115, and 117-118) displayed Ki for human M3 receptors of between about 113 and about 0.03 nM, for example, between about 15 and about 0.03 nM, or for example between about 7 and about 0.03 nM, or for example between about 0.5 and about 0.03 nM, or for example between about 0.15 and about 0.03 nM. Particular compounds (Nos. 105, 110-112, 114 and 115) displayed Ki for rat M2 receptors of between about 760 and about 24 nM, for example, between about 550 and about 24 nM, or for example between about 100 and about 24 nM, or for example between about 50 and about 24 nM. Based on such measurements, the compounds described herein displayed selectivity (Ki for human M3 receptors/Ki for human M2 receptors) of between about 1.8 and about 140, for example between about 7 and about 140, or for example between about 40 and about 140.

1. A compound having the structure of Formula I 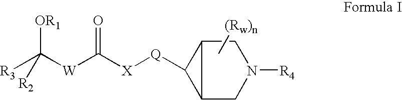
and their pharmaceutically acceptable salts, pharmaceutically acceptable solvates, esters, enantiomers, diastereomers, N-oxides, polymorphs, metabolites, wherein
R₁ is hydrogen or alkyl;
R₂ is straight or branched alkyl alkenyl, alkynyl, aryl, cycloalkyl, cycloalkylalkyl or heteroaryl wherein the alkyl, alkenyl, alkynyl, aryl, cycloalkyl, cycloalkylalkyl and heteroaryl are optionally substituted with one or more substituents selected from alkyl, hydroxy or halogen.
R₃ is aryl or heteroaryl wherein the said aryl or heteroaryl are optionally substituted with one or more groups selected from alkyl, hydroxy or halogen;
W=—(CH₂)ᵢ;
Q=—(CH₂)ⱼ;
X is oxygen or —N(R₅)—;
R₄ is hydrogen, straight or branched alkyl, straight or branched alkenyl, aralkyl or heteroarylalkyl wherein the said aralkyl or heteroarylalkyl is further substituted with alkyl, —NH₂ or alkoxycarbonylamino;
R₅ is hydrogen or alkyl;
R_{w} is H or methyl; and
n, i, j are integer from 0-2.

2. A compound selected from the group consisting of
N-(3-Benzyl-3-azabicyclo[3.1.0]hex-6-yl)-2-hydroxy-2-phenyl-2-(2-thienyl)acetamide (Compound No. 1),
N-[(3-Benzyl-3-azabicyclo[3.1.0]hex-6-yl)methyl]-2-hydroxy-2-phenyl-2-(2-thienyl)acetamide (Compound No. 2),
(3-Benzyl-3-azabicyclo[3.1.0]hex-6-yl)methyl hydroxy(phenyl)-2-thienylacetate (Compound No. 3),
(3-Benzyl-3-azabicyclo[3.1.0]hex-6-yl)methyl hydroxy(phenyl)-3-thienylacetate (Compound No. 4),
N-(3-Benzyl-3-azabicyclo[3.1.0]hex-6-yl)-2-hydroxy-2-phenyl-2-(3-thienyl)acetamide (Compound No. 5),
N-[(3-Benzyl-3-azabicyclo[3.1.0]hex-6-yl)methyl]-2-hydroxy-2-phenyl-2-(3-thienyl)acetamide (Compound No. 6),
Tartarate salt of N-[(3-benzyl-3-azabicyclo[3.1.0]hex-6-yl)methyl]-2-cyclopentyl-2-hydroxy-2-(2-thienyl)acetamide (Compound No. 7),
N-[(3-Benzyl-3-azabicyclo[3.1.0]hex-6-yl)methyl]-2-hydroxy-N-methyl-2-phenyl-2-(3-thienyl)acetamide (Compound No. 8),
Tartarate salt of N-(3-benzyl-3-azabicyclo[3.1.0]hex-6-yl)-2-cyclopentyl-2-hydroxy-2-(2-thienyl)acetamide (Compound No. 9),
N-[(3-Benzyl-3-azabicyclo[3.1.0]hex-6-yl)methyl]-2-hydroxy-2,2-di-2-thienylacetamide (Compound No. 10),
N-[(3-Benzyl-3-azabicyclo[3.1.0]hex-6-yl)methyl]-2-hydroxy-N-methyl-2-phenyl-2-(2-thienyl)acetamide (Compound No. 11),
N-[(3-Benzyl-3-azabicyclo[3.1.0]hex-6-yl)methyl]-2-cyclopentyl-2-hydroxy-N-methyl-2-(2-thienyl)acetamide (Compound No. 12),
N-[(3-Benzyl-3-azabicyclo[3.1.0]hex-6-yl)methyl]-2-hydroxy-N-methyl-2-(4-methylphenyl)-2-(2-thienyl)acetamide (Compound No. 13),
(3-Benzyl-3-azabicyclo[3.1.0]hex-6-yl)methyl hydroxy(4-methylphenyl)-2-thienylacetate (Compound No. 14),
(3-Benzyl-3-azabicyclo[3.1.0]hex-6-yl)methyl hydroxy(di-2-thienyl)acetate (Compound No. 15),
N-[(3-Benzyl-3-azabicyclo[3.1.0]hex-6-yl)methyl]-2-hydroxy-2-(4-methylphenyl)-2-(2-thienyl)acetamide (Compound No. 16),
Tartarate salt of N-(3-azabicyclo[3.1.0]hex-6-ylmethyl)-2-cyclopentyl-2-hydroxy-2-(2-thienyl)acetamide (Compound No. 17),
N-(3-Azabicyclo[3.1.0]hex-6-ylmethyl)-2-hydroxy-2-phenyl-2-(2-thienyl)acetamide (Compound No. 18),
2-Hydroxy-N-[(3-methyl-3-azabicyclo[3.1.0]hex-6-yl)methyl]-2-phenyl-2-(2-thienyl)acetamide (Compound No. 19),
N-(3-Benzyl-3-azabicyclo[3.1.0]hex-6-yl)-2-(4-fluorophenyl)-2-hydroxy-2-phenylacetamide (Compound No. 20),
N-(3-Benzyl-3-azabicyclo[3.1.0]hex-6-yl)-2-hydroxy-2-(4-methylphenyl)-2-phenylacetamide (Compound No. 21),
(3-Benzyl-3-azabicyclo[3.1.0]hex-6-yl)methyl 3-hydroxy-3,3-diphenylpropanoate (Compound No. 22),
N-[(3-Benzyl-3-azabicyclo[3.1.0]hex-6-yl)methyl]-3-hydroxy-N-methyl-3,3-diphenylpropanamide (Compound No. 23),
N-[(3-Benzyl-3-azabicyclo[3.1.0]hex-6-yl)methyl]-3-hydroxy-3,3-diphenylpropanamide (Compound No. 24),
(3-Methyl-3-azabicyclo[3.1.0]hex-6-yl)methyl hydroxy(3-methylphenyl)phenylacetate (Compound No. 25),
N-[(3-Benzyl-3-azabicyclo[3.1.0]hex-6-yl)methyl]-2-hydroxy-2-(2-hydroxy-5-methylphenyl)-2-phenylacetamide (Compound No. 26),
3-Azabicyclo[3.1.0]hex-6-ylmethyl hydroxy[bis(3-methylphenyl)]acetate (Compound No. 27),
N-(3-Azabicyclo[3.1.0]hex-6-ylmethyl)-2-hydroxy-2,2-bis(3-methylphenyl)acetamide (Compound No. 28),
N-[(3-Benzyl-3-azabicyclo[3.1.0]hex-6-yl)methyl]-2-hydroxy-2,2-bis(3-methylphenyl)acetamide (Compound No. 29),
N-(3-Azabicyclo[3.1.0]hex-6-ylmethyl)-2-hydroxy-N-methyl-2,2-bis(3-methylphenyl)acetamide (Compound No. 30),
(3-Benzyl-3-azabicyclo[3.1.0]hex-6-yl)methyl methoxy(diphenyl)acetate (Compound No. 31),
N-[(3-Benzyl-3-azabicyclo[3.1.0]hex-6-yl)methyl]-2-methoxy-2,2-diphenylacetamide (Compound No. 32),
N-(3-Benzyl-3-azabicyclo[3.1.0]hex-6-yl)-2-methoxy-2,2-diphenylacetamide (Compound No. 33),
N-[(3-Benzyl-3-azabicyclo[3.1.0]hex-6-yl)methyl]-2-methoxy-N-methyl-2,2-diphenylacetamide (Compound No. 34),
3-Azabicyclo[3.1.0]hex-6-ylmethyl methoxy(diphenyl)acetate (Compound No. 35).
N-(3-Azabicyclo[3.1.0]hex-6-ylmethyl)-3-hydroxy-3,3-diphenylpropanamide (Compound No. 36),
Tartarate salt of N-(3-azabicyclo[3.1.0]hex-6-ylmethyl)-3-hydroxy-N-methyl-3,3-diphenylpropanamide (Compound No. 37),
N-(3-Azabicyclo[3.1.0]hex-6-ylmethyl)-2-methoxy-N-methyl-2,2-diphenylacetamide (Compound No. 38),
N-3-Azabicyclo[3.1.0]hex-6-yl-2-methoxy-2,2-diphenylacetamide (Compound No. 39),
N-(3-Azabicyclo[3.1.0]hex-6-ylmethyl)-2-methoxy-2,2-diphenylacetamide (Compound No. 40),
N-(3-Azabicyclo[3.1.0]hex-6-ylmethyl)-2-methoxy-2-(4-methylphenyl)-2-phenylacetamide (Compound No. 41),
3-Azabicyclo[3.1.0]hex-6-ylmethyl diphenyl(propoxy)acetate (Compound No. 42),
N-(3-Azabicyclo[3.1.0]hex-6-ylmethyl)-N-methyl-2,2-diphenyl-2-propoxyacetamide (Compound No. 43),
N-(3-Azabicyclo[3.1.0]hex-6-ylmethyl)-2,2-diphenyl-2-propoxyacetamide (Compound No. 44),
2-Hydroxy-N-methyl-2,2-diphenyl-N-{[3-(2-thienylmethyl)-3-azabicyclo[3.1.0]hex-6-yl]methyl}acetamide (Compound No. 45),
3-Azabicyclo[3.1.0]hex-6-ylmethyl hydroxy[bis(4-methylphenyl)]acetate (Compound No. 46),
N-(3-Azabicyclo[3.1.0]hex-6-ylmethyl)-2,2-bis(4-fluorophenyl)-2-hydroxy-N-methylacetamide (Compound No. 47),
N-(3-Azabicyclo[3.1.0]hex-6-ylmethyl)-2-hydroxy-N-methyl-2,2-bis(4-methylphenyl)acetamide (Compound No. 48),
N-(3-benzyl-3-azabicyclo[3.1.0]hex-6-yl)-N-ethyl-2-hydroxy-2,2-diphenylacetamide (Compound No. 49)
2-Cyclohexyl-2-hydroxy-N-({3-[(6-methylpyridin-2-yl)methyl]-3-azabicyclo[3.1.0]hex-6-yl}methyl)-2-phenylacetamide (Compound No. 50),
N-({3-[(6-Aminopyridin-2-yl)methyl]-3-azabicyclo[3.1.0]hex-6-yl}methyl)-2-cyclopentyl-2-hydroxy-2-phenylacetamide (Compound No. 51),
N-({3-[(6-Aminopyridin-2-yl)methyl]-3-azabicyclo[3.1.0]hex-6-yl}methyl)-2-cyclohexyl-2-hydroxy-2-phenylacetamide (compound No. 52),
Tartarate salt of N-({3-[(6-aminopyridin-2-yl)methyl]-3-azabicyclo[3.1.0]hex-6-yl}methyl)-2-cyclopentyl-2-hydroxy-2-phenylacetamide (compound No. 53),
Tartarate salt of N-({3-[(6-aminopyridin-2-yl)methyl]-3-azabicyclo[3.1.0]hex-6-yl}methyl)-2-cyclohexyl-2-hydroxy-2-phenylacetamide (Compound No. 54),
2-Cycloheptyl-2-hydroxy-2-phenyl-N-{[3-(pyridin-2-ylmethyl)-3-azabicyclo[3.1.0]hex-6-yl]methyl}acetamide (Compound No. 55)
2-Cyclopentyl-2-hydroxy-N-({3-[(6-methylpyridin-2-yl)methyl]-3-azabicyclo[3.1.0]hex-6-yl}methyl)-2-phenylacetamide (Compound No. 56),
N-({3-[(6-Aminopyridin-2-yl)methyl]-3-azabicyclo[3.1.0]hex-6-yl}methyl)-2-hydroxy-2,2-diphenylacetamide (Compound No. 57),
(3-Benzyl-2-methyl-3-azabicyclo[3.1.0]hex-6-yl)methyl cyclopentyl (hydroxy)phenylacetate (Compound No. 58),
(3-Benzyl-2-methyl-3 azabicyclo[3.1.0]hex-6-yl)methyl cyclohexyl(hydroxy)phenylacetate (Compound No. 59),
N-[(3-Benzyl-2-methyl-3-azabicyclo[3.1.0]hex-6-yl)methyl]-2-cyclopentyl-2-hydroxy-2-phenylacetamide (Compound No. 60),
N-[(3-Benzyl-2-methyl-3-azabicyclo[3.1.0]hex-6-yl)methyl]-2-cyclohexyl-2-hydroxy-2-phenylacetamide (Compound No. 61),
(3-Benzyl-2-methyl-3-azabicyclo[3.1.0]hex-6-yl)methyl hydroxy(diphenyl)acetate (Compound No. 62),
Tartarate salt of N-[(3-benzyl-2-methyl-3-azabicyclo[3.1.0]hex-6-yl)methyl]-2-cyclohexyl-2-hydroxy-2-phenylacetamide (compound No. 63),
(2-Methyl-3-azabicyclo[3.1.0]hex-6-yl)methyl cyclohexyl(hydroxy)phenylacetate (Compound No. 64),
Tert-butyl (6-{[6-({[cyclohexyl(hydroxy)phenylacetyl]amino}methyl)-3-azabicyclo[3.1.0]hex-3-yl]methyl}pyridin-2-yl)carbamate (Compound No. 65),
Tert-butyl (6-{[6-({[hydroxy(diphenyl)acetyl]amino}methyl)-3-azabicyclo[3.1.0]hex-3-yl]methyl}pyridin-2-yl)carbamate (compound no. 66),
Tert-butyl (6-{[6-({[cyclopentyl(hydroxy)phenylacetyl]amino}methyl)-3-azabicyclo[3.1.0]hex-3-yl]methyl}pyridin-2-yl)carbamate (Compound No. 67),
Tartarate salt of [3-(4-methylpent-3-en-1-yl)-3-azabicyclo[3.1.0]hex-6-yl]methyl cyclopentyl(hydroxy)phenylacetate (Compound No. 68),
Tartarate salt of [3-(1-phenylethyl)-3-azabicyclo[3.1.0]hex-6-yl]methyl cyclohexyl(hydroxy)phenylacetate (Compound No. 69),
Tartarate salt of N-[(3-benzyl-3-azabicyclo[3.1.0]hex-6-yl)methyl]-2-cyclobutyl-2-hydroxy-2-phenylacetamide (Compound No. 70),
2-Hydroxy-N-{[3-(3-methylbut-2-en-1-yl)-3-azabicyclo[3.1.0]hex-6-yl]methyl}-2,2-diphenylacetamide (Compound No. 71),
2-Hydroxy-N-{[3-(4-methylpent-3-en-1-yl)-3-azabicyclo[3.1.0]hex-6-yl]methyl}-2,2-diphenylacetamide (Compound No. 72),
2-Cycloheptyl-2-hydroxy-2-phenyl-N-{[3-(pyridin-3-ylmethyl)-3-azabicyclo[3.1.0]hex-6-yl]methyl}acetamide (Compound No. 73),
2-Cycloheptyl-2-hydroxy-2-phenyl-N-{[3-(pyridin-4-ylmethyl)-3-azabicyclo[3.1.0]hex-6-yl]methyl}acetamide (Compound No. 74),
2-Cyclopentyl-2-hydroxy-N-[(3-methyl-3-azabicyclo[3.1.0]hex-6-yl)methyl]-2-phenylacetamide (Compound No. 75),
2-Cyclopentyl-2-hydroxy-2-phenyl-N-{[3-(2-phenyl ethyl)-3-azabicyclo[3.1.0]hex-6-yl]methyl}acetamide (Compound No. 76),
N-[(3-Benzyl-3-azabicyclo[3.1.0]hex-6-yl)methyl]-2-cycloheptyl-2-hydroxy-2-phenylacetamide (Compound No. 77),
N-[(3-Benzyl-3-azabicyclo[3.1.0]hex-6-yl)methyl]-2-cyclohexyl-2-hydroxy-N-methyl-2-phenylacetamide (Compound No. 78),
N-[(3-Benzyl-3-azabicyclo[3.1.0]hex-6-yl)methyl]-2-hydroxy-N-methyl-2,2-diphenylacetamide (Compound No. 79),
N-{[3-(1,3-Benzodioxol-5-ylmethyl)-3-azabicyclo[3.1.0]hex-6-yl]methyl}-2-cyclohexyl-2-hydroxy-2-phenylacetamide (Compound No. 80),
2-Cyclopentyl-2-hydroxy-2-phenyl-N-{[3-(2-thienylmethyl)-3-azabicyclo[3.1.0]hex-6-yl]methyl}acetamide (Compound No. 81),
2-Cyclohexyl-2-hydroxy-2-phenyl-N-[3-(2-phenylethyl)-3-azabicyclo[3.1.0]hex-6-yl]methyl acetamide (Compound No. 82),
N-({3-[2-(1,3-Benzodioxol-5-yl)ethyl]-3-azabicyclo[3.1.0]hex-6-yl}methyl)-2-cycloheptyl-2-hydroxy-2-phenylacetamide (Compound No. 83),
2-Cycloheptyl-N-({3-[2-(2,3-dihydro-6-benzofuran-5-yl)ethyl]-3-azabicyclo[3.1.0]hex-6-yl}methyl)-2-hydroxy-2-phenylacetamide (Compound No. 84),
N-[(3-Benzyl-3-azabicyclo[3.1.0]hex-6-yl)methyl]-2-hydroxy-3-methyl-2-phenylpentanamide (Compound No. 85),
N-[(3-Benzyl-3-azabicyclo[3.1.0]hex-6-yl)methyl]-2-cyclopentyl-2-methoxy-2-phenylacetamide (Compound No. 86),
Tartarate salt of [3-(3-methylbut-2-en-1-yl)-3-azabicyclo[3.1.0]hex-6-yl]methyl cyclopentyl(hydroxy)phenylacetate (Compound No. 87),
[3-(1,3-Benzodioxol-5-ylmethyl)-3-azabicyclo[3.1.0]hex-6-yl]methyl cyclopentyl (hydroxy)phenylacetate (Compound No. 88),
Tartarate salt of N-[(3-benzyl-3-azabicyclo[3.1.0]hex-6-yl)methyl]-2-cyclohexyl-2-hydroxy-N-methyl-2-phenylacetamide (Compound No. 89),
Tartarate salt of N-[(3-benzyl-3-azabicyclo[3.1.0]hex-6-yl)methyl]-2-hydroxy-N-methyl-2,2-diphenylacetamide (Compound No. 90),
N-[(3-Benzyl-3-azabicyclo[3.1.0]hex-6-yl)methyl]-3-cyclohexyl-2-hydroxy-2-phenylpropanamide (Compound No. 91),
2-Cyclopentyl-N-{[3 (cyclopropylmethyl)-3-azabicyclo[3.1.0]hex-6-yl]methyl}-2-hydroxy-2-phenylacetamide (Compound No. 92),
N-(3-Benzyl-3-azabicyclo[3.1.0]hex-6-yl)-2-cyclohexyl-N-ethyl-2-hydroxy-2-phenylacetamide (Compound No. 93),
N-3-azabicyclo[3.1.0]hex-6-yl-2-cyclohexyl-N-ethyl-2-hydroxy-2-phenylacetamide (Compound No. 94),
Tartarate salt of (3-benzyl-3-azabicyclo[3.1.0]hex-6-yl)methyl cyclopentyl(hydroxy)-2-thienylacetate (Compound No. 95),
N-[(3-Benzyl-3-azabicyclo[3.1.0]hex-6-yl)methyl]-3,3,3-trifluoro-2-hydroxy-2-phenylpropanamide (Compound No. 96),
(3-Benzyl-3-azabicyclo[3.1.0]hex-6-yl)methyl 2-hydroxy-2-phenylpent-4-ynoate (Compound No. 97),
N-[(3-Benzyl-3-azabicyclo[3.1.0]hex-6-yl)methyl]-2-hydroxy-2-phenylpent-4-ynamide (Compound No. 98),
[3-(1,3-Benzodioxol-5-ylmethyl)-3-azabicyclo[3.1.0]hex-6-yl]methyl cyclohexyl(hydroxy)phenylacetate (Compound No. 99),
2-Hydroxy-2,2-diphenyl-N-{[3-(2-phenylethyl)-3-azabicyclo[3.1.0]hex-6-yl]methyl}acetamide (Compound No. 100),
N-[(3-Benzyl-3-azabicyclo[3.1.0]hex-6-yl)methyl]-2-hydroxy-3-methyl-2-phenylbutanamide (Compound No. 101),
N-({3-[2-(1,3-Benzodioxol-5-yl)ethyl]-3-azabicyclo[3.1.0]hex-6-yl}methyl)-2-cyclopentyl-2-hydroxy-N-methyl-2-phenylacetamide (Compound No. 102),
Tartarate salt of N-[(3-Benzyl-3-azabicyclo[3.1.0]hex-6-yl)methyl]-2-cyclopentyl-2-hydroxy-N-methyl-2-phenylacetamide (Compound No. 103),
N-[(3-Benzyl-3-azabicyclo[3.1.0]hex-6-yl)methyl]-2-hydroxy-2-phenyl-2-pyridin-2-ylacetamide (Compound No. 104),
N-[(3-Benzyl-3-azabicyclo[3.1.0]hex-6-yl)methyl]-2-hydroxy-N-methyl-2-phenyl-2-pyridin-3-ylacetamide (Compound No. 105),
2-Hydroxy-N-methyl-N-{[3-(4-methylpent-3-en-1-yl)-3-azabicyclo[3.1.0]hex-6-yl]methyl}-2,2-diphenylacetamide (Compound No. 106),
N-({3-[2-(1,3-Benzodioxol-5-yl)ethyl]-3-azabicyclo[3.1.0]hex-6-yl}methyl)-2-hydroxy-N-methyl-2,2-diphenylacetamide (Compound No. 107),
N-({3-[2-(2,3-Dihydro-1-benzofuran-5-yl)ethyl]-3-azabicyclo[3.1.0]hex-6-yl}methyl)-2-hydroxy-N-methyl-2,2-diphenylacetamide (Compound No. 108),
N-{3-Azabicyclo[3.1.0]hex-6-ylmethyl}-N-methyl-2,2-diphenylpropanamide (Compound No. 109),
N-[(3-Benzyl-3-azabicyclo[3.1.0]hex-6-yl)methyl]-2-hydroxy-N-methyl-2-phenylhex-4-enamide (Compound No. 110),
N-[(3-Benzyl-3-azabicyclo[3.1.0]hex-6-yl)methyl]-2-hydroxy-2-phenyl-2-pyridin-3-ylacetamide (Compound No. 111),
N-[(3-Benzyl-3-azabicyclo[3.1.0]hex-6-yl)methyl]-2-hydroxy-2-phenylhex-4-enamide (Compound No. 112),
N-[(3-Benzyl-3-azabicyclo[3.1.0]hex-6-yl)methyl]-N-methyl-2,2-diphenyl-2-propoxyacetamide (Compound No. 113),
3-Azabicyclo[3.1.0]hex-6-ylmethyl (4E or 4Z)-2-hydroxy-2-phenylhex-4-enoate (Compound No. 114),
(3-Benzyl-3-azabicyclo[3.1.0]hex-6-yl)methyl (4E or 4Z)-2-hydroxy-2-phenylhex-4-enoate (Compound No. 115),
(3-Benzyl-3-azabicyclo[3.1.0]hex-6-yl)methyl (2R or 2S)-hydroxy(4-methylphenyl)phenylacetate (Compound No. 116),
(3-Benzyl-3-azabicyclo[3.1.0]hex-6-yl)methyl hydroxy(phenyl)pyridin-2-ylacetate (Compound No. 117),
(3-Benzyl-3-azabicyclo[3.1.0]hex-6-yl)methyl hydroxy(phenyl)pyridin-3-ylacetate (Compound No. 118),
N-[(3-Benzyl-3-azabicyclo[3.1.0]hex-6-yl)methyl]-N-methyl-2,2-diphenylpropanamide (Compound No. 119),
Tert-butyl 6-({[(4E)-2-hydroxy-2-phenylhex-4-enoyl]oxy}methyl)-3-azabicyclo[3.1.0]hexane-3-carboxylate (Compound No. 120).

3. A pharmaceutical composition comprising a therapeutically effective amount of a compound as defined in claim 1 together with pharmaceutically acceptable carrier, excipients or diluents.

4. A method for treatment of prophylaxis of an animal or a human suffering from a disease or disorder of the respiratory, urinary and gastrointestinal systems, wherein the disease or disorder is mediated through the muscarinic receptors, comprising administering to said animal or human, a therapeutically effective amount of a compound defined in claim 1.

5. A method according to claim 4, wherein the disease of disorder is urinary incontinence, lower urinary tract symptoms (LUTS), bronchial asthma, chronic obstructive pulmonary disease (COPD), pulmonary fibrosis, irritable bowel syndrome, obesity, diabetes, and gastrointestinal hyperkinesis.

6. A method for treatment of prophylaxis of an animal or a human suffering from a disease or disorder of the respiratory, urinary, and gastrointestinal systems, wherein the disease or disorder is mediated through the muscarinic receptors, comprising administering to said animal or human a therapeutically effective amount of a pharmaceutical composition according to claim 3.

7. (canceled)

8. (canceled)

9. (canceled)

10. A compound of claim 1, wherein
R₁ is hydrogen
R₄ is benzyl.

11. A compound of claim 10, wherein
R₂ is phenyl cycloalkyl.
